(19) United States
(12) Patent Application Publication
(10) Pub. No.: US 2023/0092359 A1
(43) Pub. Date: Mar. 23, 2023

(54) GEMFIBROZIL FORMULATION

(71) Applicant: Polaryx Therapeutics, Inc., Paramus, NJ (US)

(72) Inventor: Hahn-Jun LEE, Closter, NJ (US)

(73) Assignee: Polaryx Therapeutics, Inc., Paramus, NJ (US)

(21) Appl. No.: 17/800,498

(22) PCT Filed: Feb. 15, 2021
(86) PCT No.: PCT/US2021/018098

§ 371 Date: Aug. 17, 2022

Related U.S. Application Data
(60) Provisional application No. 62/978,375, filed on Feb. 19, 2020.

(51) Int. Cl. C07C 59/68

(52) U.S. Cl. CPC C07C 59/68; C07B 2200/13

(57) ABSTRACT

A gemfibrozil salt and composition thereof comprising a gemfibrozil salt having a solubility greater than at least that of free-form gemfibrozil in artificial saliva. A method of making a gemfibrozil salt by combining a free form of gemfibrozil with a pharmacologically acceptable salt former. A pharmaceutical composition and a method of treating a patient in need thereof with a gemfibrozil salt.

### FIELD OF INVENTION

The disclosure herein relates to providing a crystalline form of a gemfibrozil salt that is freely soluble in aqueous solutions and the methods for preparing such gemfibrozil salts.

### BACKGROUND

Gemfibrozil, or 5-(2,5-dimethylphenoxy)-2,2-dimethyl-pentanoic acid, is a fibrate compound, a class of drugs that have been shown to increase high density lipoproteins (HDL) and lower plasma triglyceride levels. Gemfibrozil may also affect other conditions such as neuronal ceroid lipofuscinosis (NCL), a neuronal lysosomal storage disease associated with abnormal accumulations of lipofuscins within cells of the CNS. The mechanism by which gemfibrozil decreases these abnormal lipofuscin accumulation is not entirely clear, but may involve peroxisome proliferator activated receptor (PPAR) α mediated pathway. Gemfibrozil may provide overall enhanced lysosomal biogenesis resulting in decreased accumulations of lipofuscins within cells of the CNS in those affected by NCL.

Gemfibrozil, is typically administered orally. But, its long hydrophobic alkyl-benzene backbone renders it poorly soluble in aqueous solutions.

The solubility of gemfibrozil in water is approximately 0.03 mg/ml compared with its solubility in organic buffers, such as ethanol and DMSO where solubilities are approximately 30 mg/ml and 16 mg/ml, respectively. Notably, solubility drastically decreases to approximately 0.5 mg/mL in a 1:1 solution of ethanol:PBS. Gemfibrozil is maximally absorbed in the upper gastrointestinal tract, but its poor solubility under acidic conditions reduces even this rate of absorption.

A gemfibrozil compound with improved solubility would most likely lead to a compound with improved dissolution when taken orally, as well as increased bioavailability.

### SUMMARY

In an aspect, the invention relates to a gemfibrozil composition comprising, consisting essentially of, or consisting of a gemfibrozil salt. The gemfibrozil salt has a solubility of approximately 247 mg/mL, 336 mg/mL, or 501 mg/mL in artificial saliva.

In an aspect, the invention relates to a method for generating a gemfibrozil salt, comprising, consisting essentially of, or consisting of dissolving or suspending a free form of gemfibrozil in a solvent and adding a pharmaceutically acceptable salt former. The pharmaceutically acceptable salt former is at least one salt former selected from the group consisting of ethanolamine, L-arginine, and L-lysine.

In an aspect, the invention relates to a pharmaceutical composition comprising, consisting essentially of, or consisting of a gemfibrozil salt. The gemfibrozil salt has a solubility of approximately 247 mg/mL, 336 mg/mL, or 501 mg/mL in artificial saliva. In an aspect, the invention relates to a pharmaceutical composition comprising, consisting essentially of, or consisting of a gemfibrozil salt prepared by a method for generating a gemfibrozil salt, comprising, consisting essentially of, or consisting of dissolving or suspending a free form of gemfibrozil in a solvent and adding a pharmaceutically acceptable salt former. The pharmaceutically acceptable salt former is at least one salt former selected from the group consisting of ethanolamine, L-arginine, and L-lysine.

In an aspect, the invention relates to a method of treating a subject having Late Infantile Neuronal Ceroid Lipofuscinosis comprising administering at least one of a gemfibrozil salt or a pharmaceutical composition comprising a gemfibrozil salt. In an aspect, the gemfibrozil salt has a solubility of approximately 247 mg/mL, 336 mg/mL, or 501 mg/mL in artificial saliva. In an aspect, the gemfibrozil salt is prepared by a method comprising, consisting essentially of, or consisting of dissolving or suspending a free form of gemfibrozil in a solvent and adding a pharmaceutically acceptable salt former. The pharmaceutically acceptable salt former is at least one salt former selected from the group consisting of ethanolamine, L-arginine, and L-lysine.

### BRIEF DESCRIPTION OF THE DRAWINGS

The following detailed description of the embodiments of the present invention will be better understood when read in conjunction with the appended drawings. For the purposes of illustration, there are shown in the drawings embodiments. It is understood, however, that the invention is not limited to the precise arrangements shown. In the drawings:

FIG. 1 Shows a dissolution curve of free-form gemfibrozil in artificial saliva.

FIG. 2 Shows an ¹H NMR spectrum of an ethanolamine gemfibrozil salt.

FIG. 3A Shows an XRPD pattern of an ethanolamine gemfibrozil salt Form A.

FIG. 3B Shows an XRPD pattern of ethanolamine gemfibrozil salt Form A prepared using a scaled-up model.

FIG. 4 Shows a dynamic vapor sorption curve of an ethanolamine gemfibrozil salt Form A across a range of relative humidities.

FIG. 5 Shows a thermogravimetric thermogram of an ethanolamine gemfibrozil salt Form A.

FIG. 6 Shows a differential scanning calorimetry thermogram of an ethanolamine gemfibrozil salt Form A.

FIG. 7A-7D Show hot stage microscopy images of an ethanolamine gemfibrozil salt Form A at various temperatures.

FIG. 8 Shows an ¹H NMR spectrum of an L-arginine gemfibrozil salt Material A.

FIG. 9 Shows an XRPD pattern of an L-arginine gemfibrozil salt Material A.

FIG. 10 Shows a dynamic vapor sorption curve of an L-arginine gemfibrozil salt Material A across a range of relative humidities.

FIG. 11A Shows an XRPD pattern of L-arginine gemfibrozil salt Material A under higher relative humidity conditions.

FIG. 11B Shows an XRPD pattern of L-arginine gemfibrozil salt Material A prepared using a scaled-up model.

FIG. 12 Shows a thermogravimetric thermogram of an L-arginine gemfibrozil salt Material A.

FIG. 13 Shows a differential scanning calorimetry thermogram of an L-arginine gemfibrozil salt Material A.

FIG. 14 Shows an ¹H NMR spectrum of an L-lysine gemfibrozil salt.

FIG. 15 Shows an XRPD pattern of an L-lysine gemfibrozil salt Material A.

FIG. 16 Shows a dynamic vapor sorption curve of an L-lysine gemfibrozil salt Material A across a range of relative humidities.

FIG. 17A Shows an XRPD pattern of an L-lysine gemfibrozil salt Material A sample after dynamic vapor sorption analysis.

FIG. 17B Shows an XRPD pattern of L-lysine gemfibrozil salt Material A prepared using a scaled-up model.

FIG. 18 Shows a thermogravimetric thermogram of an L-lysine gemfibrozil salt Material A.

FIG. 19 Shows a differential scanning calorimetry thermogram of an L-lysine gemfibrozil salt Material A.

FIG. 20A-20H Show hot stage microscopy images of an L-lysine gemfibrozil salt Material A at various temperatures.

FIG. 21 Shows an XRPD pattern and indexing solution of free-form gemfibrozil.

### DETAILED DESCRIPTION OF EMBODIMENTS

Certain terminology is used in the following description for convenience only and is not limiting. The phrase “at least one” followed by a list of two or more items such as A, B, or C, means any individual one of A, B, or C as well as any combination thereof. Terms such as “approximately,” “about,” “substantially” are construed as modifying terms defined by the circumstances and as understood by those skilled in the art. The degree of modification includes at least the degree of expected experimental error.

Embodiments include a gemfibrozil composition comprising, consisting essentially of, or consisting of a gemfibrozil salt having a solubility of greater than free form gemfibrozil in an aqueous solution. In an embodiment, the gemfibrozil salt has a solubility of greater than free form gemfibrozil in at least water, a base, and a physiological solution. In an embodiment, the gemfibrozil salt has a solubility of approximately 247 mg/ml in artificial saliva. In an embodiment, the gemfibrozil salt has a solubility of approximately 336 mg/ml in artificial saliva. In an embodiment, the gemfibrozil salt has a solubility of approximately 501 mg/ml in artificial saliva. In an embodiment, the gemfibrozil may have a solubility in aqueous solution of 100 mg/ml to 200 mg/ml, 200 mg/ml to 300 mg/ml, 300 mg/ml to 400 mg/ml, 400 mg/ml to 500 mg/ml, 500 mg/ml to 600 mg/ml, 600 mg/ml to 700 mg/ml, 700 mg/ml to 800 mg/ml, 800 mg/ml to 900 mg/ml, 900 mg/ml to 1000 mg/ml, or any interval between any two of these solubility range endpoints (for example 100 mg/ml to 1000 mg/ml, or 300 mg/ml to 800 mg/ml). In an embodiment, the gemfibrozil salt may have a solubility in aqueous solution selected from 5 mg/ml increments in the range of 100 mg/ml to 1000 mg/ml (that is, the solubility in aqueous solution may be 100 mg/ml, 105 mg/ml, 110 mg/ml . . . 990 mg/ml, 995 mg/ml, or 1000 mg/ml). In an embodiment, the gemfibrozil salt may have a solubility in aqueous solution in a range of 100 mg/ml to 1000 mg/ml, or in a sub-range thereof. The sub-range low endpoint may be selected from 100 mg/ml, 105 mg/ml, 110 mg/ml . . . 990 mg/ml, or 995 mg/ml, while the sub-rang high endpoint may be selected from 105 mg/ml, 110 mg/ml, 115 mg/ml . . . 995 mg/ml or 1000 mg/ml, where the high endpoint selected is higher than the low endpoint selected). The solubility values may be determined visually as approximate values.

In an embodiment, artificial saliva is an aqueous solution that mimics physiological saliva. In an embodiment, artificial saliva may mimic mammalian saliva. In an embodiment, artificial saliva may mimic human saliva. In an embodiment, the artificial saliva is an aqueous solution having a pH of approximately 7.3 and comprises, consists essentially of, or consists of at least one of potassium chloride, sodium chloride, sodium bicarbonate, alpha-amylase, and mucin gastric. In an embodiment, artificial saliva comprises, consists essentially of, or consists of 0.0150% potassium chloride, 0.0117% sodium chloride, 0.2105% sodium bicarbonate, 0.2003% alpha-amylase, and 0.1020% mucin gastric in an aqueous solution having a pH of approximately 7.3.

In an embodiment, a gemfibrozil salt may have at least one of the following properties in this paragraph. The gemfibrozil salt may be freely soluble in an aqueous solution. The gemfibrozil salt may be more soluble than free gemfibrozil in an aqueous solution. The gemfibrozil salt may be freely soluble in artificial saliva. The gemfibrozil salt may rapidly dissolve in an aqueous solution. The gemfibrozil salt may rapidly dissolve in artificial saliva. The gemfibrozil salt may have a 1:1 mol:mol stoichiometry of gemfibrozil:salt former. The gemfibrozil salt may have a single crystalline phase. The gemfibrozil salt may be in a crystalline phase having a degree of disorder. The gemfibrozil salt may have a consistent XRPD pattern following a stress. The gemfibrozil salt may have a low hygroscopicity. The gemfibrozil salt may be anhydrous. The gemfibrozil salt may be unsolvated. The gemfibrozil salt may have the property of not experiencing weight loss greater than 0.1% at temperatures below approximately 125° C. The gemfibrozil salt may have the property of not experiencing an onset of decomposition below temperatures of approximately 140° C. The gemfibrozil salt may have a melting temperature of at least approximately 59° C. Specific examples of a gemfibrozil salt are discussed below. Each may have one or more of the properties listed in this paragraph.

In an embodiment, the gemfibrozil salt may be an ethanolamine gemfibrozil salt, an L-arginine gemfibrozil salt, a L-lysine gemfibrozil salt, a sodium gemfibrozil salt, a potassium gemfibrozil salt, or a piperazine gemfibrozil salt. In an embodiment, the gemfibrozil salt may be an ethanolamine gemfibrozil salt, an L-arginine gemfibrozil salt, or a L-lysine gemfibrozil salt.

In an embodiment, the gemfibrozil salt may be an ethanolamine gemfibrozil salt. Unless otherwise stated, an ethanolamine gemfibrozil salt having Ethanolamine Form A is referenced in the examples provided herein.

In an embodiment, the solubility of an ethanolamine gemfibrozil salt may be demonstrated by a visual solubility assessment. An example of a visual solubility assessment follows. For example, an amount of an ethanolamine gemfibrozil salt, ethanolamine gemfibrozil salt Form A, was suspended in a pH 7.3 artificial saliva solution. For the ethanolamine gemfibrozil salt Form A, this was approximately 10.0 mg of the salt. The solution was then mixed by vortexing or sonicating, until complete dissolution was observed. As shown in Table 1 below, 0.02 ml of artificial saliva was added until the solution appeared clear, with no solids present, indicating that the ethanolamine gemfibrozil salt Form A has a solubility of approximately 501 mg/ml in artificial saliva, and that the ethanolamine gemfibrozil salt Form A is freely soluble as defined in the solubility descriptors presented herein.

TABLE 1

| | | | Volume of | | | |
|---|---|---|---|---|---|---|
| | | Weight of | Media | | | |
| | | compound | Added | Solubility | Solubility | |
| NB# | Gemfibrozil | (mg) | (mL) | (mg/mL) | Descriptor * | Observations |
| 7724-18-01 | Lysine | 10.09 | 0.03 | 336.33 | freely | Clear liquid |
| | Material A | | | | soluble | |
| 7724-18-02 | Arginine | 9.89 | 0.04 | 247.25 | freely | Clear liquid |
| | Material A | | | | soluble | |
| 7724-18-03 | Ethanolamine | 10.02 | 0.02 | 501 | freely | Clear liquid |
| | Form A | | | | soluble | |
| 7724-18-04 | Free Form | 100.32 | 94.8 | <1.1 | very slightly | Cloudy liquid |
| | | | | | soluble | with solids |
| | | | | | | present |

In an embodiment, the ethanolamine gemfibrozil salt Form A may have a solubility as listed above for a gemfibrozil salt. In an embodiment, the ethanolamine gemfibrozil salt Form A may have a solubility of 100 mg/ml to 200 mg/ml; 200 mg/ml to 300 mg/ml; 300 mg/ml to 400 mg/ml; 400 mg/ml to 500 mg/ml; 500 mg/ml to 600 mg/ml; 600 mg/ml to 700 mg/ml; 700 mg/ml to 800 mg/ml; 800 mg/ml to 900 mg/ml; 900 mg/ml to 1000 mg/ml; or any interval between any of these solubility ranges. In an embodiment, the ethanolamine gemfibrozil salt Form A may have a solubility selected from 50 mg/ml increments selected from the range of 100 mg/ml to 1000 mg/ml.

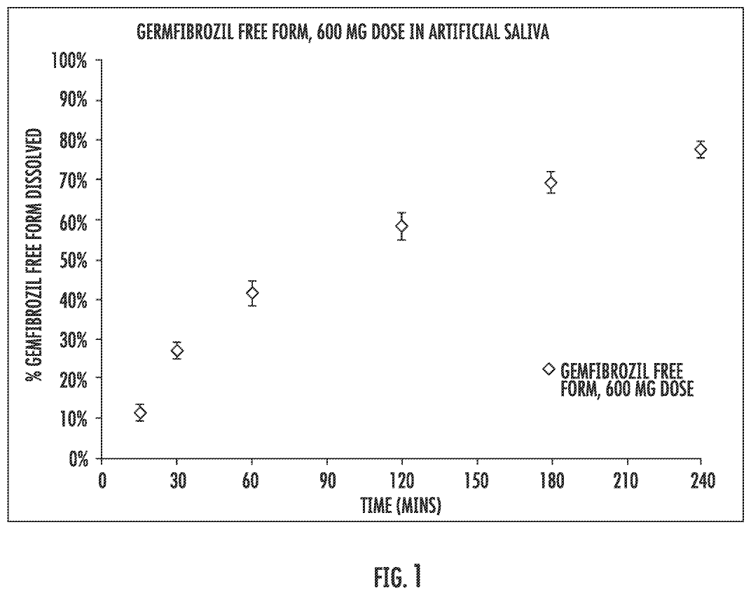

In contrast, free-form gemfibrozil is only very slightly soluble. For example, 100 mg of free-form gemfibrozil was suspended in a pH 7.3 artificial saliva solution as shown in Table 1. The solution was then mixed by vortexing or sonication, but the solution remained cloudy with solids present even upon addition of 95 ml of artificial saliva, indicating that free-form gemfibrozil has a solubility of less than 1.1 mg/ml. In another example, as shown in FIG. 1, even after 4 hours of dissolution time, whereby 600 mg of free-form gemfibrozil were suspended in 900 mL of pH 7.3 artificial saliva, with stirring at 100 rpm, at 37° C., an average of only 78% of the free-form gemfibrozil dissolved completely.

In an embodiment, an ethanolamine gemfibrozil salt may be freely soluble in an aqueous solution. In an embodiment, the solubility of an ethanolamine gemfibrozil salt may be demonstrated by a visual solubility assessment. An example of a visual solubility assessment follows. For example, approximately 1499 mg of the ethanolamine gemfibrozil Form A salt was suspended in 1.5 ml of pH 7.3 artificial saliva, and stirred for 16 hours. As shown in Table 2 below, no excess solids remained indicating that the equilibrium solubility of the ethanolamine gemfibrozil Form A salt is greater than 999 mg/ml. In an embodiment, the equilibrium solubility of the ethanolamine gemfibrozil salt may be 900 mg/ml to 1000 mg/ml; 1000 mg/ml to 1100 mg/ml; 1100 mg/ml to 1200 mg/ml; 1200 mg/ml to 1500 mg/ml; 1500 mg/ml to 2000 mg/ml; or any interval between any of these equilibrium solubility ranges. In an embodiment, the ethanolamine gemfibrozil salt may have an equilibrium solubility selected from 50 mg/ml increments selected from the range of 900 mg/ml to 2000 mg/ml.

TABLE 2

| | Weight of | | | Solubility |
|---|---|---|---|---|
| | compound | | Solubility | Descriptor |
| Gemfibrozil | (mg) | Observations | (mg/mL) | * |
| Lysine | 801.50 | No excess solids | >534.33 | freely |
| Material A | | remaining, light | | soluble |
| | | yellow solution | | |
| Arginine | 700.50 | No excess solids | >467.00 | freely |
| Material A | | remaining, clear | | soluble |
| | | solution | | |
| Ethanolamine | 1499.40 | No excess solids | >999.60 | freely |
| Form A | | remaining, | | soluble |
| | | cloudy solution | | |

In an example, the solutions were diluted and filtered using a 0.45 μm Nylon membrane, in order to assess both filtered and unfiltered solutions. As shown in Table 3 below, the approximate equilibrium solubility concentration of the ethanolamine gemfibrozil salt Form A in the filtered solution is approximately 647 mg/ml and that of the unfiltered solution is approximately 583 mg/ml.

TABLE 3

| | | | | PARK | | | | |
|---|---|---|---|---|---|---|---|---|
| | | File | | Area | Concentration | Dilution | Solubility | |
| | Description | Name | RT | (m Au*s) | (mg/mL) | factor | (mg/mL) | Page |
| Filtered | Lysine | 949069 | 9.89 | 412.22 | 0.08 | 2666.67 | >337.67 | 47 |
| | Material A | | | | | | | |
| | Arginine | 949070 | 9.89 | 372.25 | 0.07 | 2666.67 | >326.47 | 48 |
| | Material A | | | | | | | |
| | Ethanolamine | 949071 | 9.89 | 469.02 | 0.09 | 5714.29 | >646.57 | 49 |
| | Form A | | | | | | | |
| Unfiltered | Lysine | 949072 | 9.87 | 400.77 | 0.08 | 2666.67 | >328.29 | 50 |
| | Material A | | | | | | | |
| | Arginine | 949073 | 9.88 | 353.92 | 0.07 | 2666.67 | >310.39 | 51 |
| | Material A | | | | | | | |
| | Ethanolamine | 949074 | 9.87 | 423.20 | 0.08 | 5714.29 | >583.40 | 52 |
| | Form A | | | | | | | |

An ethanolamine gemfibrozil salt may be rapidly dissolving. In an embodiment, greater than 95% of the ethanolamine gemfibrozil salt Form A dissolves within 5 minutes when added to an aqueous solution. For example, an amount of ethanolamine gemfibrozil salt Form A equivalent to approximately 100 mg of free-form gemfibrozil was suspended in 250 ml of a pH 7.3 artificial saliva solution at 37° C. At time points of 5, 10, and 15 minutes, approximately 2 ml aliquots of the solution were filtered, and the filtrate was analyzed by HPLC in order to determine the concentration of the ethanolamine gemfibrozil salt present. As shown in Table 4 below, 97.3% of the ethanolamine gemfibrozil salt Form A dissolved within 5 minutes of incubation. The amount of the ethanolamine gemfibrozil salt Form A that dissolved at 15 minutes was approximately the same as at 5 minutes.

TABLE 4

| | | Average % Gemfibrozil |
|---|---|---|
| Description | Time point (min) | dissolved |
| Arginine Material | 5 | 96.64% |
| A | 10 | 94.40% |
| | 15 | 94.27% |
| Ethanolamine Form | 5 | 97.30% |
| A | 10 | 94.80% |
| | 15 | 95.84% |
| Lysine Material A | 5 | 96.29% |
| | 10 | 93.51% |
| | 15 | 94.67% |

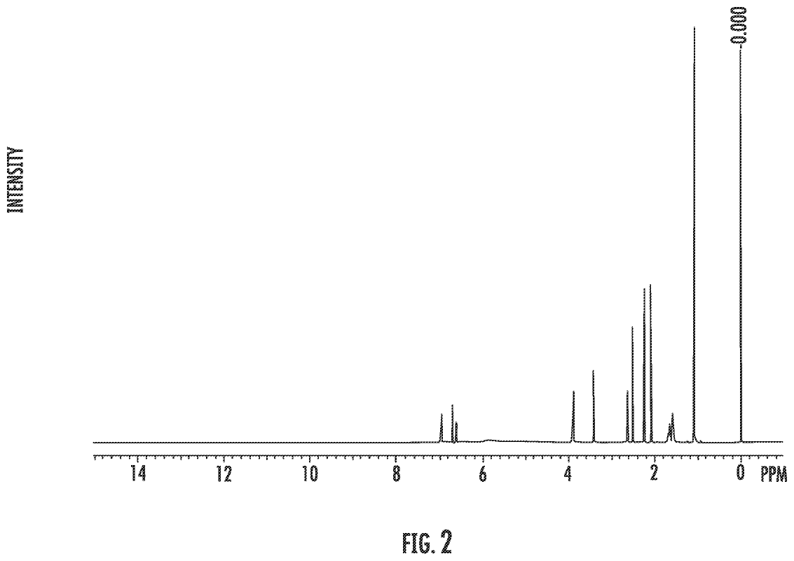

In an embodiment, ethanolamine gemfibrozil salt Form A has a 1:1 mol:mol stoichiometry of gemfibrozil:ethanolamine as determined by solution proton nuclear magnetic resonance spectroscopy (¹H NMR). As illustrated in FIG. 2, the ¹H NMR spectrum of ethanolamine gemfibrozil salt Form A is consistent with the chemical structure of gemfibrozil, based on the peak of API at 1.1 ppm (attributable to the gem-dimethyl protons), and the salt former, based on the peaks at 3.4 and 2.6 ppm (attributable to the methylene protons) with a 1:1 stoichiometry. The ¹H NMR spectrum of FIG. 2 is also consistent with the absence of organic solvents.

In an embodiment, a single unique XRPD pattern was produced for an ethanolamine gemfibrozil salt. This was designated Ethanolamine Form A. An XRPD pattern and an XRPD indexing solution indicate that it is composed of a single crystalline phase. In an embodiment, the ethanolamine gemfibrozil salt Form A has a triclinic unit cell containing two molecules of gemfibrozil. The volume of the unit cell, calculated from the indexing solution, may be consistent with one molecule of ethanolamine per molecule of gemfibrozil and may not indicate room for solvent.

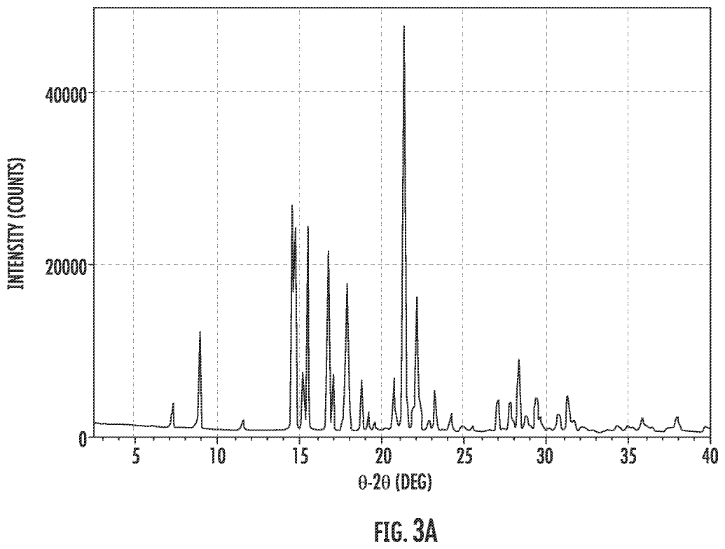

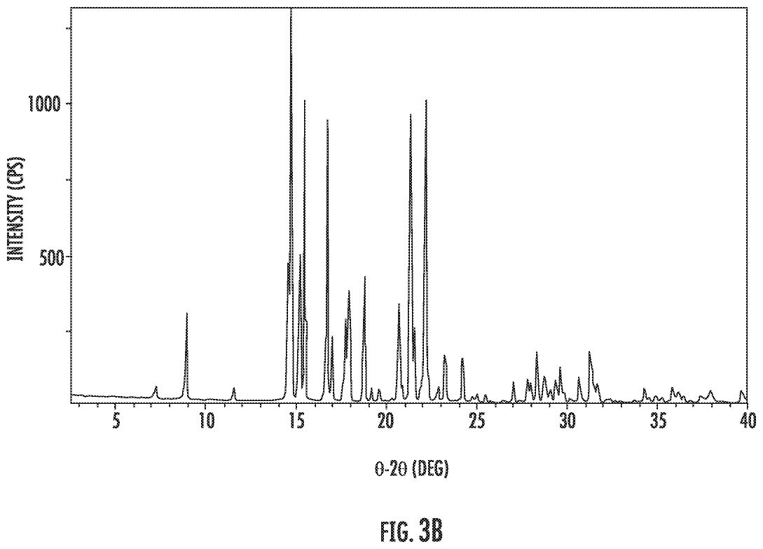

Referring to FIG. 3A, an embodiment of an ethanolamine gemfibrozil salt may have an XRPD pattern comprising observed peaks at angular position 2θ as shown in Table 5, below. Prominent observed peaks for the ethanolamine gemfibrozil salt Form A salt are found at angular positions 8.94+/−0.2, 14.5+/−0.2, 14.70+/−0.2, 15.20+/−0.2, 15.49+/−0.2, 16.72+/−0.2, 17.01+/−0.2, 17.97+/−0.2, 21.34+/−0.2, and 22.14+/−0.2. The representative peaks or characteristic peaks of the ethanolamine gemfibrozil salt Form A may vary from the observed peaks of this example.

TABLE 5

| °2θ | d space (Å) | Intensity (%) |
|---|---|---|
| 7.26 ± 0.20 | 12.170 ± 0.335 | 7 |
| 8.94 ± 0.20 | 9.884 ± 0.221 | 23 |
| 11.55 ± 0.20 | 7.658 ± 0.132 | 4 |
| 14.55 ± 0.20 | 6.081 ± 0.083 | 46 |
| 14.70 ± 0.20 | 6.020 ± 0.081 | 54 |
| 15.20 ± 0.20 | 5.824 ± 0.076 | 20 |
| 15.49 ± 0.20 | 5.716 ± 0.073 | 58 |
| 16.72 ± 0.20 | 5.299 ± 0.063 | 52 |
| 17.01 ± 0.20 | 5.209 ± 0.061 | 17 |
| 17.60 ± 0.20 | 5.035 ± 0.057 | 5 |
| 17.76 ± 0.20 | 4.991 ± 0.056 | 13 |
| 17.91 ± 0.20 | 4.948 ± 0.055 | 30 |
| 18.79 ± 0.20 | 4.720 ± 0.050 | 18 |
| 19.20 ± 0.20 | 4.619 ± 0.048 | 6 |
| 19.61 ± 0.20 | 4.524 ± 0.046 | 4 |
| 20.29 ± 0.20 | 4.373 ± 0.043 | 2 |
| 20.71 ± 0.20 | 4.286 ± 0.041 | 17 |
| 20.87 ± 0.20 | 4.253 ± 0.040 | 6 |
| 21.34 ± 0.20 | 4.161 ± 0.039 | 100 |
| 21.52 ± 0.20 | 4.127 ± 0.038 | 12 |
| 21.91 ± 0.20 | 4.054 ± 0.037 | 6 |
| 22.14 ± 0.20 | 4.012 ± 0.036 | 41 |
| 22.29 ± 0.20 | 3.985 ± 0.035 | 9 |
| 22.88 ± 0.20 | 3.884 ± 0.033 | 4 |
| 23.24 ± 0.20 | 3.825 ± 0.032 | 11 |
| 24.21 ± 0.20 | 3.673 ± 0.030 | 7 |
| 24.81 ± 0.20 | 3.586 ± 0.028 | 3 |
| 25.02 ± 0.20 | 3.556 ± 0.028 | 3 |
| 25.49 ± 0.20 | 3.491 ± 0.027 | 3 |
| 26.51 ± 0.20 | 3.360 ± 0.025 | 2 |
| 27.05 ± 0.20 | 3.294 ± 0.024 | 9 |
| 27.37 ± 0.20 | 3.256 ± 0.023 | 2 |
| 27.82 ± 0.20 | 3.205 ± 0.023 | 9 |
| 27.97 ± 0.20 | 3.188 ± 0.022 | 4 |
| 28.32 ± 0.20 | 3.149 ± 0.022 | 19 |
| 28.72 ± 0.20 | 3.106 ± 0.021 | 5 |
| 28.82 ± 0.20 | 3.095 ± 0.021 | 5 |
| 29.05 ± 0.20 | 3.071 ± 0.021 | 3 |
| 29.39 ± 0.20 | 3.037 ± 0.020 | 9 |
| 29.64 ± 0.20 | 3.011 ± 0.020 | 6 |

In an example, the amount of ethanolamine gemfibrozil salt Form A was scaled-up during preparation. In an embodiment, the amount of ethanolamine gemfibrozil salt Form A was produced on at least a one gram scale. As illustrated in FIG. 3B, the observed peaks by XRPD of the scaled-up salt were consistent with the pattern of peaks for smaller scale production of ethanolamine gemfibrozil salt Form A (FIG. 3A). These data suggest that upon scaling up the amount of the salt produced, the same ethanolamine gemfibrozil salt Form A is formed as evidenced by the lack of form change determined by XRDP analysis.

In an embodiment, ethanolamine gemfibrozil salt Form A has a low hygroscopicity below approximately 55% relative humidity, meaning less than 0.5% increases in weight below approximately 55% relative humidity, which may be determined using dynamic vapor sorption (DVS) to detect changes in weight of the salt in response to changes in relative humidity. For example, following equilibration of the salt at 5% relative humidity, the relative humidity was increased incrementally to 95% (sorption), and subsequently decreased (desorption) back to 5% relative humidity. Table 6 lists the percent change in weight of ethanolamine gemfibrozil salt Form A (Sorp Mass Change (%)) in 10% relative humidity increments from its initial weight at 5% relative humidity to its weight at 95% relative humidity.

TABLE 6

| | | | Sorp | | Desorp | |
|---|---|---|---|---|---|---|
| | Target | Sample | Mass | Sample | Mass | |
| | RH | RH | Change | RH | Change | Hyster- |
| | (%) | (%) | (%) | (%) | (%) | esis |
| Cycle 1 | 5.0 | 4.4 | 0.00 | 4.7 | 1.92 | |
| | 15.0 | 15.1 | 0.00 | 15.4 | 2.60 | 2.60 |
| | 25.0 | 24.2 | 0.00 | 24.5 | 3.56 | 3.56 |
| | 35.0 | 33.0 | 0.01 | 33.7 | 4.88 | 4.87 |
| | 45.0 | 44.3 | 0.03 | 44.9 | 6.70 | 6.67 |
| | 55.0 | 54.3 | 0.10 | 54.8 | 9.29 | 9.19 |
| | 65.0 | 64.5 | 11.14 | 64.8 | 12.93 | 1.78 |
| | 75.0 | 74.4 | 17.93 | 74.6 | 18.56 | 0.64 |
| | 85.0 | 84.4 | 27.33 | 84.7 | 31.49 | 4.16 |
| | 95.0 | 95.6 | 45.05 | 95.6 | 45.05 | |

As described above in Table 6, and as illustrated in FIG. 4, following equilibration of the ethanolamine gemfibrozil salt Form A at 5% relative humidity, the percent change in mass of the ethanolamine gemfibrozil salt Form A remained steady until about 55% relative humidity, and then increased as the relative humidity increased (FIG. 4—Diamonds). From 5% to 55% relative humidity, the percent increase in mass of the ethanolamine gemfibrozil salt Form A was approximately 0.1% as determined using DVS. However, from a relative humidity of 55% to 95%, the percent change in mass of the ethanolamine gemfibrozil salt Form A was 44.9% demonstrating the highly hygroscopic nature of the salt at these elevated relative humidities. This increase in percent mass may be even greater because the sample did not equilibrate within the time limits for these steps. FIG. 4 also demonstrates that following desorption from 95% to 5% relative humidity (FIG. 4—Squares), the ethanolamine gemfibrozil salt Form A retained approximately 2% of the gained moisture. Upon desorption hysteresis was observed, particularly from 55% to 25% relative humidity. In this example, the post-DVS sample deliquesced.

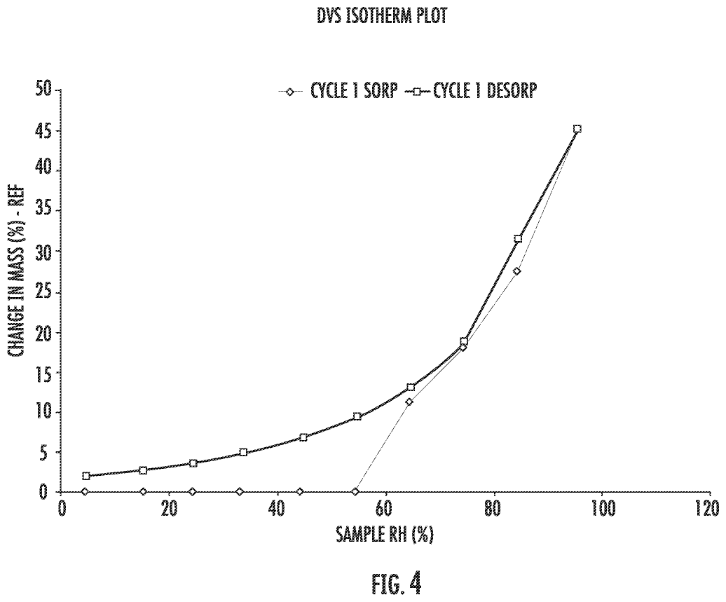

In an embodiment, an ethanolamine gemfibrozil salt is substantially anhydrous and unsolvated, which may be determined using thermogravimetric analysis (TGA). An exemplary ethanolamine gemfibrozil salt, ethanolamine gemfibrozil salt Form A, does not experience weight loss at temperatures below 140° C. As illustrated in FIG. 5, the weight of ethanolamine gemfibrozil salt Form A does not decrease until approximately 140° C. as determined by TGA. This temperature may be the onset of decomposition.

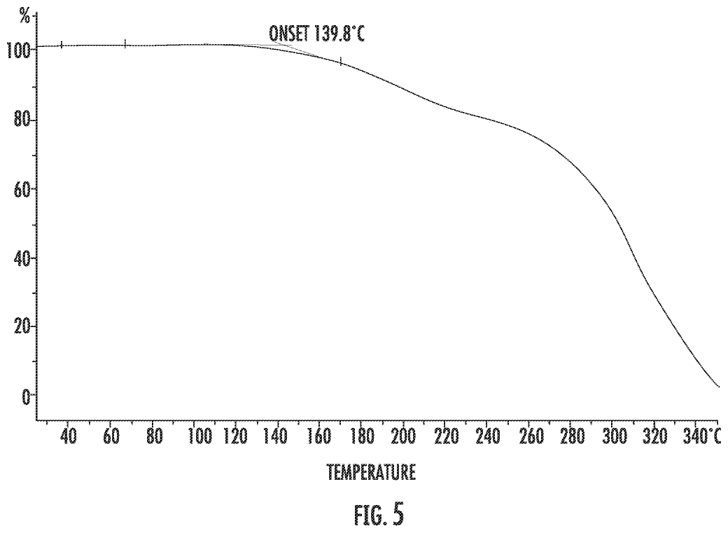

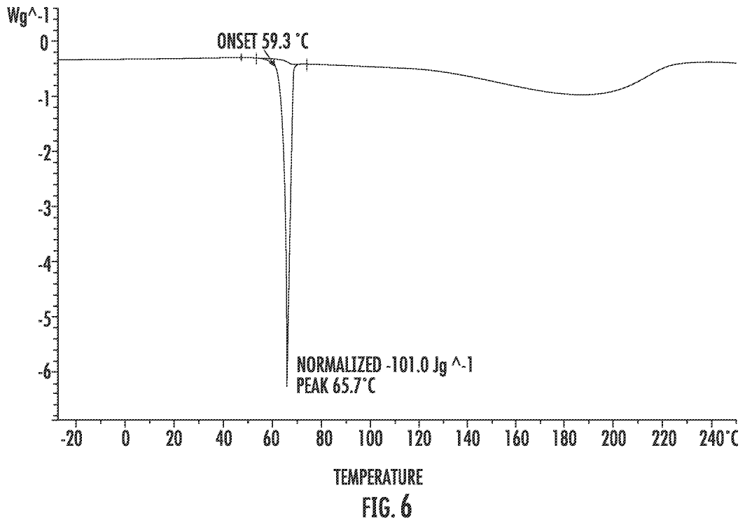

In an embodiment, ethanolamine gemfibrozil salt Form A has a melting temperature from 59° C. to 66° C. In an embodiment, the onset of melting may occur at approximately 59° C. and is observed as a sharp endotherm during differential scanning calorimetry (DSC). The DSC thermogram shown in FIG. 6 illustrates that an exemplary ethanolamine gemfibrozil salt Form A has a maximum endotherm peak at 66° C.

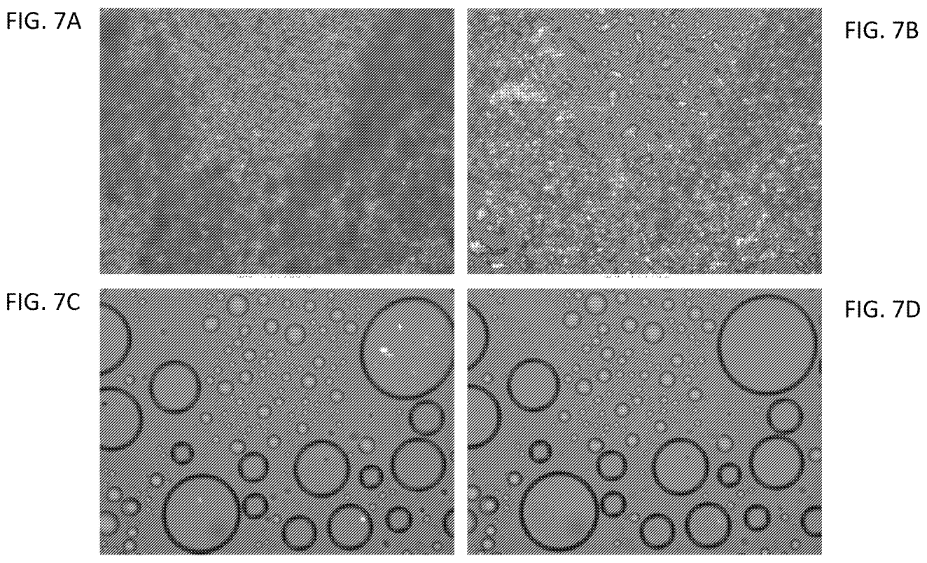

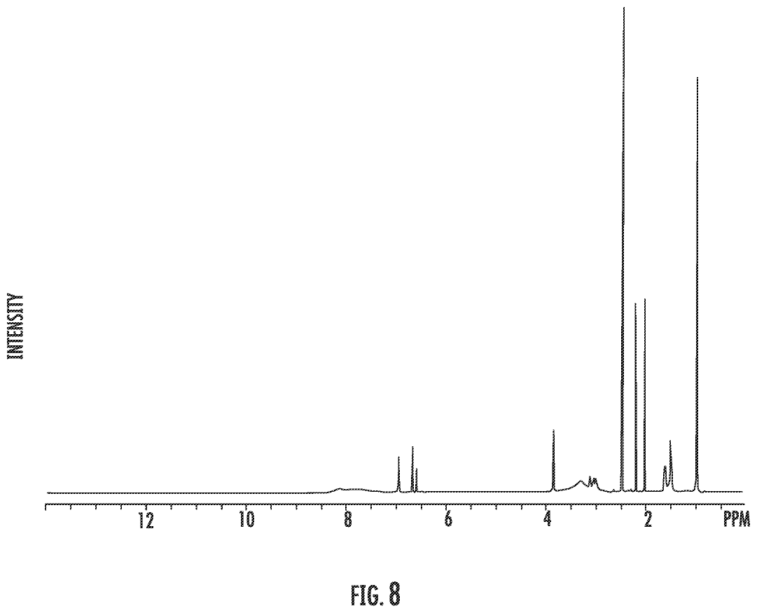

In an embodiment, an ethanolamine gemfibrozil salt has a melting temperature starting at approximately 62.9° C. as determined by hot-stage microscopy. FIG. 7A illustrates ethanolamine gemfibrozil salt Form A at ambient temperature. FIG. 7B illustrates the exemplary ethanolamine gemfibrozil salt at 62.9° C., where the image indicates that melting has begun. FIG. 7C illustrates a fully melted ethanolamine gemfibrozil salt Form A sample by 65.2° C. FIG. 7D illustrates that upon re-cooling, crystals of ethanolamine gemfibrozil salt Form A do not form.

In an embodiment, a gemfibrozil ethanolamine salt may have at least one of the following properties, including being freely soluble in an aqueous solution as determined by the equilibrium solubility values presented in Table 2 for the gemfibrozil ethanolamine salt Form A; having a solubility of approximately 501 mg/ml in artificial saliva; having a solubility of approximately selected from 50 mg/ml increments selected from the range of 100 mg/ml to 1000 mg/ml; rapidly dissolve in an aqueous solution; rapidly dissolve in an aqueous solution as described in Table 4; having an equilibrium solubility selected from 50 mg/ml increments selected from the range of 900 mg/ml to 2000 mg/ml; being more soluble in an aqueous solution than free-form gemfibrozil; having a 1:1 mol:mol stoichiometry of gemfibrozil:ethanolamine as determined by a ¹H NMR spectrum of the ethanolamine gemfibrozil salt; having a single crystalline phase; having a single unique XRPD pattern; having an XRPD pattern comprising observed peaks at angular position 2θ as shown in Table 5; (although the representative peaks or characteristic peaks of the ethanolamine gemfibrozil salt may vary from the observed peaks of this example); having a consistent XRPD pattern between small-batch production versus scaled-up production of the gemfibrozil ethanolamine salt; having a low hygroscopicity below approximately 55% relative humidity; having a low hygroscopicity below 55% relative humidity and experiencing increases in mass of approximately 0.1% between 5% to 55% relative humidity as determined using DVS; having a hygroscopic nature above 55% relative humidity; being substantially anhydrous and unsolvated; being substantially anhydrous and unsolvated as determined by TGA; not experiencing weight loss at temperatures below 140° C. as determined by TGA; having a melting temperature from 59° C. to 66° C.; having an onset of melting occur at approximately 59° C. as determined by having a sharp endotherm during DSC; having a melting temperature starting at approximately 62.9° C. as determined by hot-stage microscopy; and being an ethanolamine gemfibrozil salt as described for ethanolamine gemfibrozil salt Form A.

In an embodiment the gemfibrozil salt may be an I-arginine gemfibrozil salt. Unless otherwise stated, an L-arginine gemfibrozil salt having Arginine Material A is referenced in the examples provided herein.

In an embodiment, the solubility of an L-arginine gemfibrozil salt may be demonstrated by a visual solubility assessment. An example of a visual solubility assessment follows. For example, an amount of an L-arginine gemfibrozil salt, L-arginine gemfibrozil Material A, was suspended in a pH 7.3 artificial saliva solution. For the L-arginine gemfibrozil salt Material A, this was approximately 9.9 mg of the salt. The solution was then mixed by vortexing or sonicating, until complete dissolution was observed. As shown in Table 1 above, 0.04 ml of artificial saliva was added until the solution appeared clear, with no solids present, indicating that L-arginine gemfibrozil salt Material A has a solubility of approximately 247 mg/ml in artificial saliva, and that the L-arginine gemfibrozil salt Material A is freely soluble. This is in contrast to the much lower solubility of the free-form gemfibrozil discussed above.

In an embodiment, the L-arginine gemfibrozil salt Material A may have a solubility as listed above for a gemfibrozil salt. In an embodiment, the L-arginine gemfibrozil salt Material A may have a solubility of 100 mg/ml to 200 mg/ml; 200 mg/ml to 300 mg/ml; 300 mg/ml to 400 mg/ml; 400 mg/ml to 500 mg/ml; 500 mg/ml to 600 mg/ml; 600 mg/ml to 700 mg/ml; 700 mg/ml to 800 mg/ml; 800 mg/ml to 900 mg/ml; 900 mg/ml to 1000 mg/ml; or any interval between any of these solubility ranges. In an embodiment, the L-arginine gemfibrozil salt Material A may have a solubility selected from 50 mg/ml increments selected from the range of 100 mg/ml to 1000 mg/ml.

In an embodiment, an L-arginine gemfibrozil salt may be freely soluble in an aqueous solution. In an embodiment, the solubility of an L-arginine gemfibrozil salt may be demonstrated by a visual solubility assessment. An example of a visual solubility assessment follows. For example, approximately 701 mg of an the L-arginine gemfibrozil salt Material A was suspended in 1.5 ml of pH 7.3 artificial saliva, and stirred for 16 hours. As shown in Table 2 above, no excess solids remained indicating that the equilibrium solubility of the ethanolamine gemfibrozil salt Material A is greater than 467 mg/ml. In an embodiment, the equilibrium solubility of the L-arginine gemfibrozil salt may be 300 mg/ml to 400 mg/ml; 400 mg/ml to 500 mg/ml; 500 mg/ml to 600 mg/ml; 600 mg/ml to 700 mg/ml; 700 mg/ml to 800 mg/ml; 800 mg/ml to 900 mg/ml; 900 mg/ml to 1000 mg/ml; or any interval between any of these equilibrium solubility ranges. In an embodiment, the L-arginine gemfibrozil salt may have an equilibrium solubility selected from 50 mg/ml increments selected from the range of 300 mg/ml to 1000 mg/ml.

In an example, the solutions were diluted and filtered using a 0.45 μm Nylon membrane, in order to assess both filtered and unfiltered solutions. As shown in Table 3 above, the approximate equilibrium solubility concentration of the L-arginine gemfibrozil salt Material A in the filtered solution is approximately 326 mg/ml and that of the unfiltered solution is approximately 310 mg/ml.

An L-arginine gemfibrozil salt may be rapidly dissolving. In an embodiment, greater than 95% of the L-arginine gemfibrozil salt Material A dissolved within 5 minutes when added to an aqueous solution. For example, an amount of L-arginine gemfibrozil salt material A equivalent to approximately 100 mg of free-form gemfibrozil was suspended in 250 ml of a pH 7.3 artificial saliva solution at 37° C. At time points of 5, 10, and 15 minutes, 2 ml aliquots of the solution was filtered, and the filtrate was analyzed by HPLC in order to determine the concentration of the L-arginine gemfibrozil salt present. As shown in Table 4 above, 96.6% of the L-arginine gemfibrozil salt Material A dissolved within 5 minutes of incubation. The amount of the L-arginine gemfibrozil salt Material A that dissolved at 15 minutes was approximately the same as at 5 minutes.

In an embodiment, L-arginine gemfibrozil salt Material A has a 1:1 mol:mol stoichiometry of gemfibrozil:arginine as determined by solution proton nuclear magnetic resonance spectroscopy (¹H NMR). As illustrated in FIG. 8, the ¹H NMR spectrum of L-arginine gemfibrozil salt Material A is consistent with the chemical structure of gemfibrozil, based on the peak of API at 2.1 and 2.2 ppm (attributable to the protons of the methyl groups on the phenyl ring), and the salt former, based on the peaks at 3.0-3.2 and 1.5-1.7 ppm (attributable to the methylene protons) with a 1:1 stoichiometry. The ¹H NMR spectra of FIG. 8 is also consistent with the absence of organic solvents.

In an embodiment, two materials were found to exhibit unique XRPD patterns for an L-arginine gemfibrozil salt. These were designated Arginine Material A and Arginine Material B. In an embodiment, a single material was found and exhibited a unique XRPD pattern for an L-arginine gemfibrozil salt. This was designated Arginine Material A. In an embodiment, a method used to form an L-arginine gemfibrozil salt resulted in a single form of an L-arginine gemfibrozil salt. In an embodiment, an L-arginine gemfibrozil salt is a crystalline mono-arginine salt of gemfibrozil. In an embodiment, the XRPD pattern was unable to be indexed.

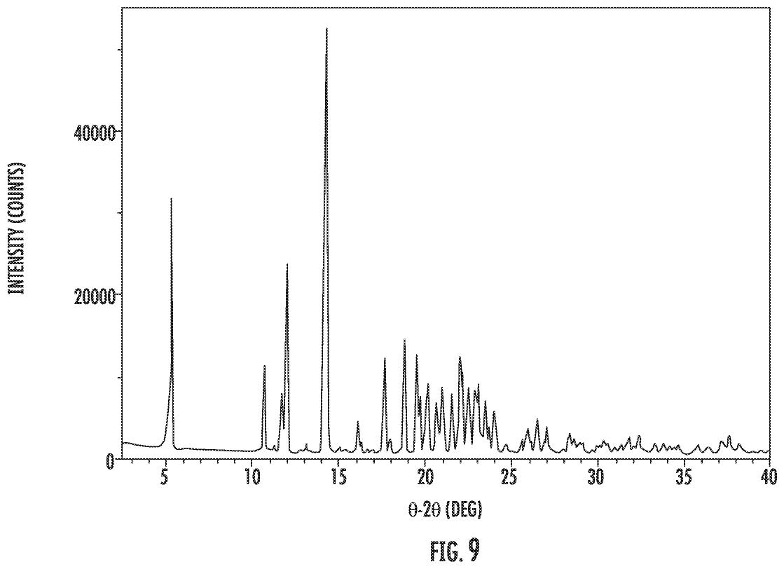

Referring to FIG. 9, an embodiment of an L-arginine gemfibrozil salt may have an XRPD pattern comprising observed peaks at angular position 2θ as shown in Table 7 below. Prominent observed peaks for the Iarginine gemfibrozil salt Material A are found at angular positions 5.36+/−0.2, 10.74+/−0.2, 12.02+/−0.2, 14.32+/−0.2, 17.69+/−0.2, 18.86+/−0.2, and 19.55+/−0.2. The representative peaks or characteristic peaks of the Iarginine gemfibrozil salt Material A may vary from the observed peaks of this example.

TABLE 7

| °2θ | d space (Å) | Intensity (%) |
|---|---|---|
| 5.36 ± 0.20 | 16.469 ± 0.614 | 50 |
| 10.74 ± 0.20 | 8.231 ± 0.153 | 18 |
| 11.28 ± 0.20 | 7.840 ± 0.139 | 3 |
| 11.70 ± 0.20 | 7.559 ± 0.129 | 12 |
| 11.80 ± 0.20 | 7.496 ± 0.127 | 16 |
| 12.02 ± 0.20 | 7.355 ± 0.122 | 48 |
| 12.84 ± 0.20 | 6.889 ± 0.107 | 2 |
| 13.17 ± 0.20 | 6.719 ± 0.102 | 4 |
| 14.32 ± 0.20 | 6.182 ± 0.086 | 100 |
| 15.10 ± 0.20 | 5.864 ± 0.077 | 5 |
| 15.46 ± 0.20 | 5.726 ± 0.074 | 2 |
| 16.16 ± 0.20 | 5.481 ± 0.067 | 8 |
| 16.31 ± 0.20 | 5.429 ± 0.066 | 4 |
| 16.67 ± 0.20 | 5.315 ± 0.063 | 2 |
| 17.69 ± 0.20 | 5.011 ± 0.056 | 22 |
| 18.00 ± 0.20 | 4.924 ± 0.054 | 5 |
| 18.86 ± 0.20 | 4.700 ± 0.049 | 32 |
| 19.55 ± 0.20 | 4.537 ± 0.046 | 28 |
| 19.70 ± 0.20 | 4.502 ± 0.045 | 17 |
| 20.12 ± 0.20 | 4.409 ± 0.043 | 16 |
| 20.20 ± 0.20 | 4.392 ± 0.043 | 19 |
| 20.69 ± 0.20 | 4.289 ± 0.041 | 13 |
| 21.03 ± 0.20 | 4.220 ± 0.040 | 15 |
| 21.60 ± 0.20 | 4.111 ± 0.038 | 14 |
| 22.07 ± 0.20 | 4.024 ± 0.036 | 23 |
| 22.20 ± 0.20 | 4.001 ± 0.036 | 23 |
| 22.57 ± 0.20 | 3.937 ± 0.034 | 18 |
| 22.96 ± 0.20 | 3.870 ± 0.033 | 15 |
| 23.12 ± 0.20 | 3.844 ± 0.033 | 17 |
| 23.51 ± 0.20 | 3.781 ± 0.032 | 15 |
| 23.73 ± 0.20 | 3.747 ± 0.031 | 8 |
| 24.03 ± 0.20 | 3.701 ± 0.030 | 11 |
| 24.72 ± 0.20 | 3.599 ± 0.029 | 4 |
| 25.13 ± 0.20 | 3.541 ± 0.028 | 2 |
| 25.65 ± 0.20 | 3.470 ± 0.027 | 5 |
| 25.98 ± 0.20 | 3.427 ± 0.026 | 8 |
| 26.51 ± 0.20 | 3.360 ± 0.025 | 10 |
| 26.89 ± 0.20 | 3.313 ± 0.024 | 5 |
| 27.09 ± 0.20 | 3.289 ± 0.024 | 7 |
| 28.05 ± 0.20 | 3.178 ± 0.022 | 3 |
| 28.40 ± 0.20 | 3.140 ± 0.022 | 6 |
| 28.64 ± 0.20 | 3.115 ± 0.021 | 5 |

In an embodiment, L-arginine gemfibrozil salt Material A has a low hygroscopicity below approximately 85% relative humidity, meaning less than 1% increases in weight below approximately 85% relative humidity, which may be determined using DVS to detect changes in weight of the salt in response to changes in relative humidity. For example, following equilibration of the salt at 5% relative humidity, the relative humidity was increased incrementally to 95% (sorption), and subsequently decreased (desorption) back to 5% relative humidity. Table 8 lists the percent change in weight of I-arginine gemfibrozil salt Material A (Sorp Mass Change (%) in 10% relative humidity increments from its initial weight at 5% relative humidity to 95% relative humidity.

TABLE 8

| | | | Sorp | | Desorp | |
|---|---|---|---|---|---|---|
| | Target | Sample | Mass | Sample | Mass | |
| | RH | RH | Change | RH | Change | Hyster- |
| | (%) | (%) | (%) | (%) | (%) | esis |
| Cycle 1 | 5.0 | 4.2 | 0.00 | 4.5 | 5.55 | |
| | 15.0 | 14.9 | 0.03 | 15.3 | 6.12 | 6.09 |
| | 25.0 | 24.1 | 0.06 | 24.4 | 6.85 | 6.79 |
| | 35.0 | 33.0 | 0.16 | 33.5 | 7.89 | 7.73 |
| | 45.0 | 44.3 | 0.25 | 44.7 | 9.51 | 9.27 |
| | 55.0 | 54.2 | 0.28 | 54.7 | 11.75 | 11.46 |
| | 65.0 | 64.2 | 0.35 | 64.8 | 14.95 | 14.60 |
| | 75.0 | 74.1 | 0.50 | 74.7 | 19.47 | 18.97 |
| | 85.0 | 83.9 | 0.83 | 84.7 | 26.79 | 25.96 |
| | 95.0 | 95.7 | 45.96 | 95.7 | 45.96 | |

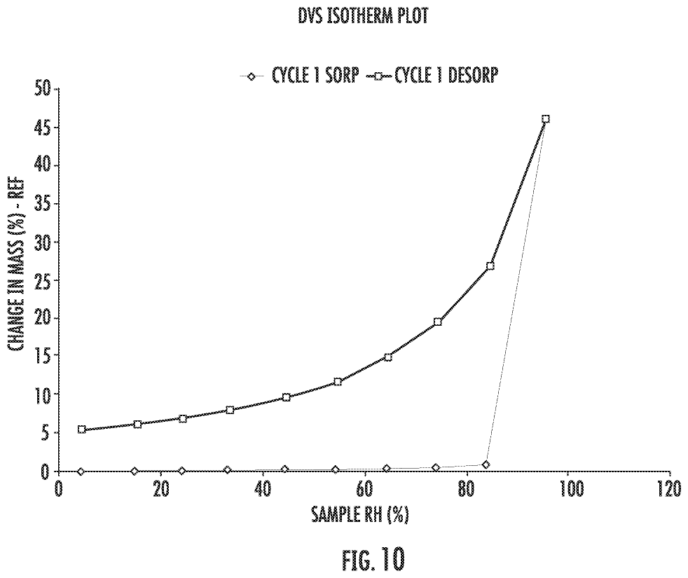

As described above in Table 8, and as illustrated in FIG. 10, following equilibration of the Iarginine gemfibrozil salt Material A at 5% relative humidity, the percent change in mass of the L-arginine gemfibrozil salt Material A only slightly increased until about 85% relative humidity, and then increased dramatically as the relative humidity increased (FIG. 10—Diamonds). From 5% to 85% relative humidity, the percent increase in mass of the L-arginine gemfibrozil salt Material A is approximately 0.8% as determined using DVS. However, from a relative humidity of 85% to 95%, the percent change in mass of the L-arginine gemfibrozil salt Material A was 45.1%, demonstrating the highly hygroscopic nature of the salt at these elevated relative humidities. This increase in percent mass could even be greater because the sample did not equilibrate within the time limits for these steps. FIG. 10 also demonstrates that following desorption from 95% to 5% relative humidity (FIG. 10—Squares), L-arginine gemfibrozil salt Material A retains approximately 5.6% of the gained moisture. Upon desorption, hysteresis is observed. In this example, the post-DVS sample deliquesced.

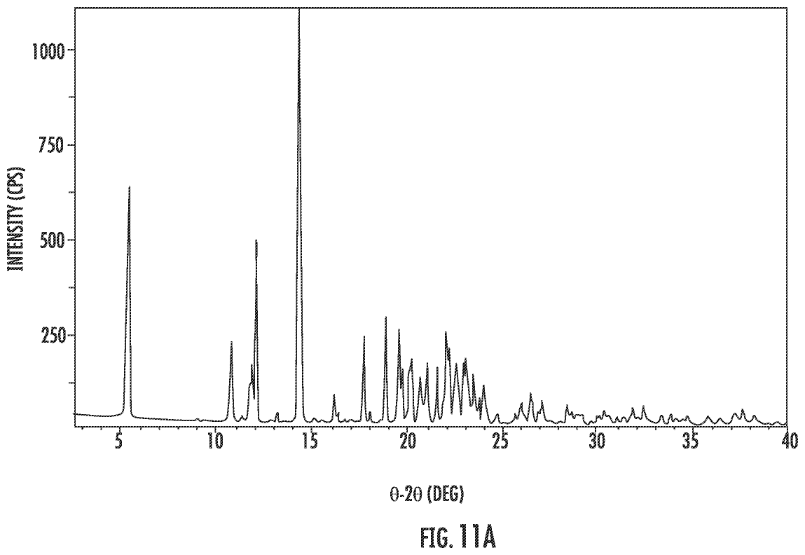

In an example, an L-arginine gemfibrozil salt was stressed for one day at 75% relative humidity. As illustrated in FIG. 11A, the observed peaks by XRPD of the stressed salt were consistent with the pattern of peaks for an L-arginine gemfibrozil salt prior to such stress (FIG. 9). These data suggest that under these stressed conditions, the exemplary L-arginine gemfibrozil salt did not undergo a form change.

In an example, the amount of L-arginine gemfibrozil salt Material A was scaled-up during preparation. In an embodiment, the amount of L-arginine gemfibrozil salt Material A was produced on at least a one gram scale. As illustrated in FIG. 11B, the observed peaks by XRPD of the scaled-up salt were consistent with the pattern of peaks for smaller scale production of L-arginine gemfibrozil salt Material A (FIG. 9). These data suggest that upon scaling up the amount of the salt produced, the same L-arginine gemfibrozil salt Material A is formed as evidenced by the lack of form change determined by XRDP analysis.

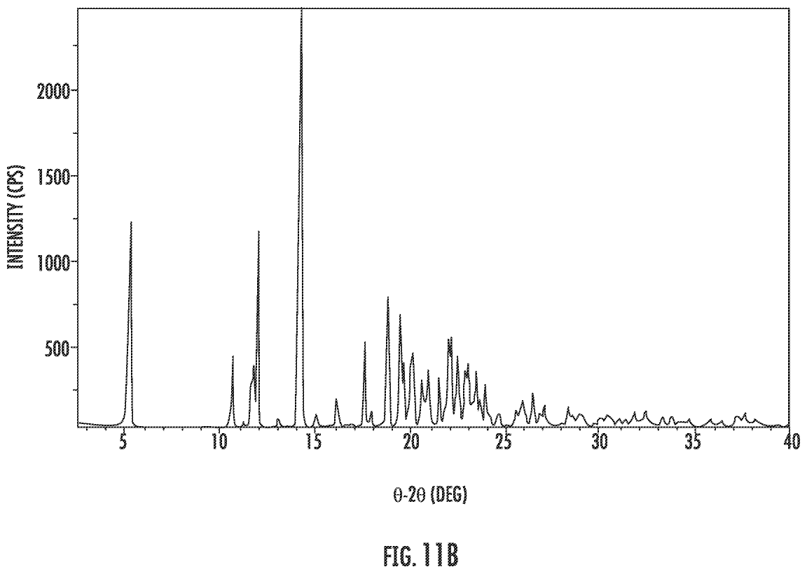

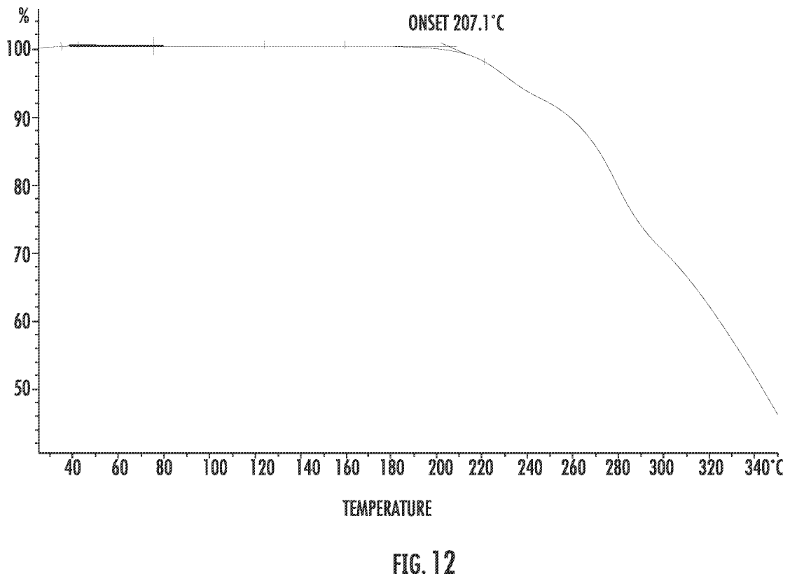

In an embodiment, an L-arginine gemfibrozil salt is substantially anhydrous and unsolvated, which may be determined using TGA. An exemplary L-arginine gemfibrozil salt, L-arginine gemfibrozil salt Material A, experiences negligible weight loss of approximately 0.1% at temperatures between 44° C. and 125° C. as demonstrated by TGA. The negligible weight loss may be the result of residual water loss given that no organic solvents were observed by NMR as shown in FIG. 8. As illustrated in FIG. 12, the weight of L-arginine gemfibrozil salt Material A does not decrease until approximately 207° C. as determined by TGA. This temperature may be the onset of decomposition.

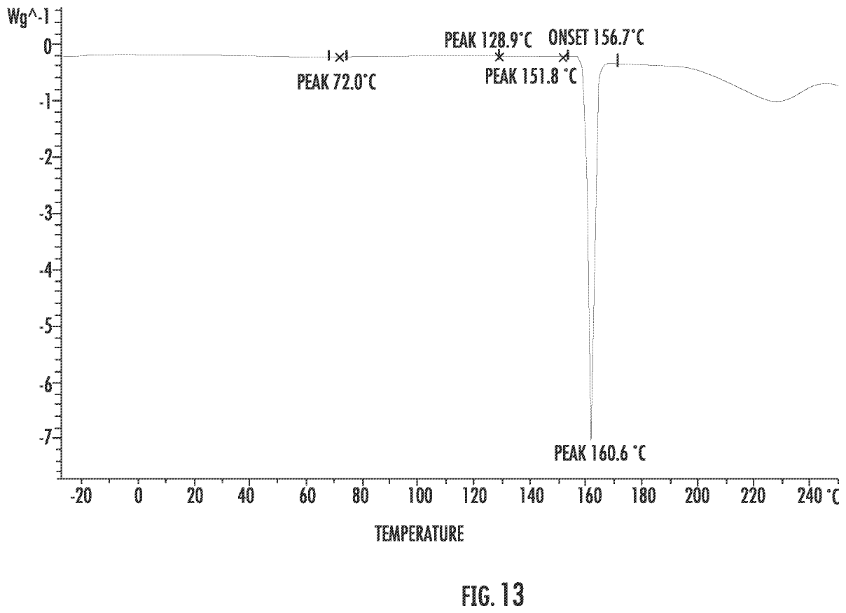

In an embodiment, L-arginine gemfibrozil salt Material A has a large endotherm as determined by DSC, which may coincide with the melting temperature of the salt. The exemplary salt has broad endotherm peaks at 72° C., 129° C., and 152° C. as illustrated in FIG. 13. The largest endotherm peak has an onset at approximately 157° C. that peaks at approximately 161° C., which may coincide with the melting temperature of the salt

In an embodiment, a gemfibrozil L-arginine salt may have at least one of the following properties, including being freely soluble in an aqueous solution as determined by the equilibrium solubility values presented in Table 2 for the gemfibrozil L-arginine salt Material A; having a solubility of approximately 247 mg/ml in artificial saliva; having a solubility selected from 50 mg/ml increments selected from the range of 100 mg/ml to 1000 mg/ml; rapidly dissolve in an aqueous solution; rapidly dissolve in an aqueous solution as described in Table 4; having an equilibrium solubility selected from 50 mg/ml increments selected from the range of 300 mg/ml to 1000 mg/ml; being more soluble in an aqueous solution than free-form gemfibrozil; having a 1:1 mol:mol stoichiometry of gemfibrozil:L-arginine as determined by a ¹H NMR spectrum of the I-arginine gemfibrozil salt; having a single crystalline phase; having a single unique XRPD pattern; having an XRPD pattern comprising observed peaks at angular position 2θ as shown in Table 7 (although the representative peaks or characteristic peaks of the I-arginine gemfibrozil salt may vary from the observed peaks of this example); having a consistent XRPD pattern between small-batch production versus scaled-up production of the gemfibrozil L-arginine salt; having a low hygroscopicity below approximately 85% relative humidity; having a low hygroscopicity below 85% relative humidity and experiencing increases in mass of approximately 0.8% between 5% to 85% relative humidity as determined using DVS; having a highly hygroscopic nature above between 85% to 95% relative humidity; having no change in form upon exposure to increased humidity as determined by DVS; having a consistent XRPD pattern between a gemfibrozil L-arginine salt following one day at 75% relative humidity versus the gemfibrozil I-arginine salt prior to any increases in relative humidity as described for the DVS experiments disclosed in Table 8 for example; being substantially anhydrous and unsolvated; being substantially anhydrous and unsolvated as determined by TGA; not experiencing weight loss of approximately greater than 0.1% between 44° C. and 125° C. as determined by TGA; not experiencing substantial weight loss until approximately 207° C.; not experiencing weight loss of greater than 1% until approximately 207° C.; having an onset of decomposition at approximately 207° C.; having a melting temperature from approximately 157° C. to 161° C.; having an onset of melting occur at approximately 152° C. as determined by having a sharp endotherm during DSC; having a peak endoderm during DSC at approximately 162° C.; having broad endotherm peaks at 72° C., 129° C., and 152° C. as determined by DSC; and being an L-arginine gemfibrozil salt as described for L-arginine gemfibrozil salt Material A.

In an embodiment the gemfibrozil salt may be an L-lysine gemfibrozil salt. Unless otherwise stated, an L-lysine gemfibrozil salt having L-lysine Material A is referenced in the examples provided herein.

In an embodiment, the solubility of an L-lysine gemfibrozil salt may be demonstrated by a visual solubility assessment. An example of a visual solubility assessment follows. For example, an amount of an L-lysine gemfibrozil salt, L-lysine gemfibrozil Material A was suspended in a pH 7.3 artificial saliva solution. For an L-lysine gemfibrozil salt Material A, this was approximately 10.1 mg of the salt. The solution was then mixed by vortexing or sonicating, until complete dissolution was observed. As shown in Table 1 above, 0.03 ml of artificial saliva was added until the solution appeared clear, with no solids present, indicating that the L-lysine gemfibrozil salt Material A has a solubility of approximately 336 mg/ml in artificial saliva, and that the L-lysine gemfibrozil salt Material A is freely soluble. This is in contrast to the much lower solubility of the free-form gemfibrozil discussed above.

In an embodiment, the L-lysine gemfibrozil salt Form A may have a solubility as listed above for a gemfibrozil salt. In an embodiment, the L-lysine gemfibrozil salt may have a solubility of 100 mg/ml to 200 mg/ml; 200 mg/ml to 300 mg/ml; 300 mg/ml to 400 mg/ml; 400 mg/ml to 500 mg/ml; 500 mg/ml to 600 mg/ml; 600 mg/ml to 700 mg/ml; 700 mg/ml to 800 mg/ml; 800 mg/ml to 900 mg/ml; 900 mg/ml to 1000 mg/ml; or any interval between any of these solubility ranges. In an embodiment, the L-lysine gemfibrozil salt may have a solubility selected from 50 mg/ml increments selected from the range of 100 mg/ml to 1000 mg/ml.

In an embodiment, an L-lysine gemfibrozil salt may be freely soluble in an aqueous solution. In an embodiment, the solubility of an L-arginine gemfibrozil salt may be demonstrated by a visual solubility assessment. An example of a visual solubility assessment follows. For example, approximately 802 mg of the L-lysine gemfibrozil salt Material A was suspended in 1.5 ml of pH 7.3 artificial saliva, and stirred for 16 hours. As shown in Table 2 above, no excess solids remained indicating that the equilibrium solubility of an L-lysine gemfibrozil salt Material A is greater than 534 mg/ml. In an embodiment, the equilibrium solubility of the L-lysine gemfibrozil salt may be 400 mg/ml to 500 mg/ml; 500 mg/ml to 600 mg/ml; 600 mg/ml to 700 mg/ml; 700 mg/ml to 800 mg/ml; 800 mg/ml to 900 mg/ml; 900 mg/ml to 1000 mg/ml; or any interval between any of these equilibrium solubility ranges. In an embodiment, the L-lysine gemfibrozil salt may have an equilibrium solubility selected from 50 mg/ml increments selected from the range of 400 mg/ml to 1000 mg/ml.

In an example, the solutions were diluted and filtered using a 0.45 μm Nylon membrane, in order to assess both filtered and unfiltered solutions. As shown in Table 3 above, the approximate equilibrium solubility concentrations of an L-lysine gemfibrozil salt Material A in the filtered solution are approximately 338 mg/ml and that of the unfiltered solution are approximately 328 mg/ml.

An L-lysine gemfibrozil salt may be rapidly dissolving. In an embodiment, greater than 95% of the L-lysine gemfibrozil salt Material A dissolves within 5 minutes when added to an aqueous solution. For example, an amount of L-lysine gemfibrozil salt Material A equivalent to approximately 100 mg of free-form gemfibrozil was suspended in 250 ml of a pH 7.3 artificial saliva solution at 37° C. At time points of 5, 10, and 15 minutes, approximately 2 ml aliquots of the solution were filtered, and the filtrate was analyzed by HPLC in order to determine the concentration of the gemfibrozil salt present. As shown in Table 4 above, 96.3% of L-lysine gemfibrozil salt Material A dissolved within 5 minutes of incubation. The amount of L-lysine gemfibrozil salt dissolved at 15 minutes was approximately the same as at 5 minutes.

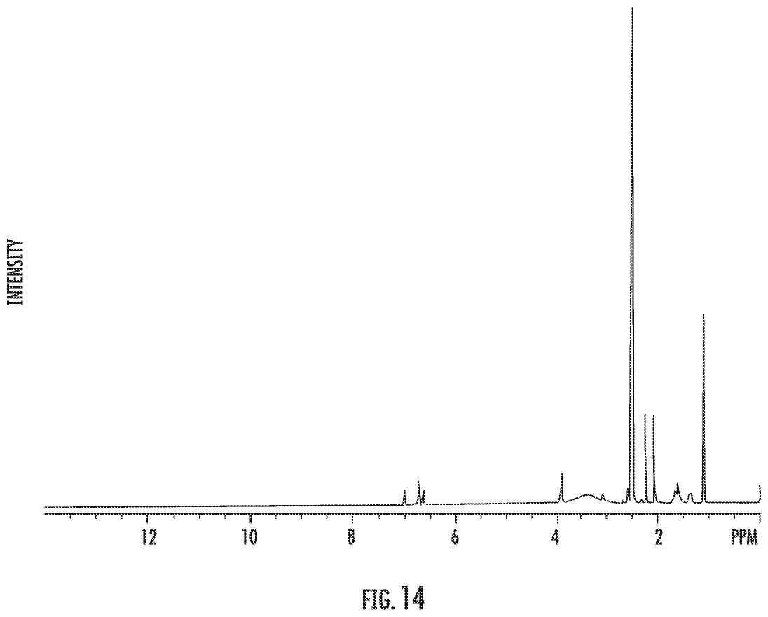

In an embodiment, the L-lysine gemfibrozil salt has a 1:1 mol:mol stoichiometry of gemfibrozil:L-lysine as determined by solution proton nuclear magnetic resonance spectroscopy (¹H NMR). As illustrated in FIG. 14, the ¹H NMR spectrum of L-lysine gemfibrozil salt Material A is consistent with the chemical structure of gemfibrozil, based on the peak of API at 2.1 and 2.2 ppm (attributable to the protons of the methyl groups on the phenyl ring), and the salt former, based on the peaks at 3.1, 2.6, 1.6, and 1.4 ppm (attributable to the methylene protons) with 1:1 stoichiometry. The ¹H NMR spectra of FIG. 14 is also consistent with the absence of organic solvents.

In an embodiment, a single unique XRPD pattern was produced for an L-lysine gemfibrozil salt. This was designated Lysine Material A.

In an embodiment, a method used to form an L-lysine gemfibrozil salt resulted in a single form of an L-lysine gemfibrozil salt. In an embodiment, an L-lysine gemfibrozil salt is a crystalline lysine salt of gemfibrozil having some degree of disorder. In an embodiment, the XRPD pattern was unable to be indexed, perhaps due to the observed disorder, perhaps due to a mixture of crystalline phases, or perhaps for other reasons.

Referring to FIG. 15, an embodiment of an L-lysine gemfibrozil salt may have an XRPD pattern comprising observed peaks at angular position 2θ as shown in Table 9 below. Prominent observed peaks for this salt are found at angular positions 5.36+/−0.2, 10.74+/−0.2, 12.02+/−0.2, 14.32+/−0.2, 17.69+/−0.2, 18.86+/−0.2, and 19.55+/−0.2. The representative peaks or characteristic peaks of the L-lysine gemfibrozil salt Material A may vary from the observed peaks of this example.

TABLE 9

| °2θ | dspace (Å) | Intensity (%) |
|---|---|---|
| 3.36 ± 0.20 | 26.303 ± 1.567 | 100 |
| 6.74 ± 0.20 | 13.103 ± 0.388 | 17 |
| 9.52 ± 0.20 | 9.280 ± 0.194 | 4 |
| 10.13 ± 0.20 | 8.728 ± 0.172 | 4 |
| 10.67 ± 0.20 | 8.282 ± 0.155 | 7 |
| 13.50 ± 0.20 | 6.554 ± 0.097 | 37 |
| 13.70 ± 0.20 | 6.456 ± 0.094 | 12 |
| 14.13 ± 0.20 | 6.265 ± 0.088 | 15 |
| 14.72 ± 0.20 | 6.014 ± 0.081 | 11 |
| 15.49 ± 0.20 | 5.716 ± 0.073 | 43 |
| 16.39 ± 0.20 | 5.405 ± 0.066 | 14 |
| 16.94 ± 0.20 | 5.230 ± 0.061 | 6 |
| 17.42 ± 0.20 | 5.086 ± 0.058 | 33 |
| 18.54 ± 0.20 | 4.782 ± 0.051 | 7 |
| 19.14 ± 0.20 | 4.634 ± 0.048 | 68 |
| 19.43 ± 0.20 | 4.565 ± 0.047 | 24 |
| 19.75 ± 0.20 | 4.491 ± 0.045 | 19 |
| 20.31 ± 0.20 | 4.369 ± 0.043 | 20 |
| 21.04 ± 0.20 | 4.219 ± 0.040 | 33 |
| 21.43 ± 0.20 | 4.143 ± 0.038 | 10 |
| 21.70 ± 0.20 | 4.093 ± 0.037 | 9 |
| 22.49 ± 0.20 | 3.951 ± 0.035 | 14 |
| 23.52 ± 0.20 | 3.780 ± 0.032 | 7 |
| 23.78 ± 0.20 | 3.739 ± 0.031 | 19 |
| 23.99 ± 0.20 | 3.706 ± 0.030 | 11 |
| 24.49 ± 0.20 | 3.631 ± 0.029 | 9 |
| 25.23 ± 0.20 | 3.527 ± 0.028 | 6 |
| 25.66 ± 0.20 | 3.468 ± 0.027 | 12 |
| 26.76 ± 0.20 | 3.329 ± 0.024 | 6 |
| 27.21 ± 0.20 | 3.275 ± 0.024 | 17 |
| 27.64 ± 0.20 | 3.225 ± 0.023 | 9 |
| 28.48 ± 0.20 | 3.131 ± 0.022 | 9 |
| 29.32 ± 0.20 | 3.044 ± 0.020 | 4 |
| 29.73 ± 0.20 | 3.003 ± 0.020 | 5 |

In an embodiment, an L-lysine gemfibrozil salt Material A has a low hygroscopicity below approximately 55% relative humidity, meaning less than 2% increases in weight below approximately 55% relative humidity, which may be determined using DVS to detect changes in weight of the salt in response to changes in relative humidity. For example, following equilibration of the salt at 5% relative humidity, the relative humidity was increased incrementally to 95% (sorption), and subsequently decreased (desorption) back to 5% relative humidity. Table 10 lists the percent change in weight of L-lysine gemfibrozil salt Material A (Sorp Mass Change (%)) in 10% relative humidity increments from its initial weight at 5% relative humidity to 95% relative humidity.

TABLE 10

| | | | Sorp | | Desorp | |
|---|---|---|---|---|---|---|
| | Target | Sample | Mass | Sample | Mass | |
| | RH | RH | Change | RH | Change | Hyster- |
| | (%) | (%) | (%) | (%) | (%) | esis |
| Cycle 1 | 5.0 | 6.2 | 0.00 | 4.7 | 2.09 | |
| | 15.0 | 15.2 | 0.04 | 15.6 | 2.53 | 2.48 |
| | 25.0 | 24.2 | 0.19 | 24.6 | 2.81 | 3.61 |
| | 35.0 | 33.0 | 0.40 | 33.9 | 3.09 | 2.69 |
| | 45.0 | 44.3 | 0.91 | 45.0 | 3.45 | 2.54 |
| | 55.0 | 54.2 | 1.79 | 54.9 | 4.09 | 2.30 |
| | 65.0 | 64.3 | 3.15 | 64.9 | 5.25 | 2.09 |
| | 75.0 | 74.2 | 6.32 | 74.6 | 9.49 | 3.16 |
| | 85.0 | 84.4 | 14.48 | 84.7 | 27.95 | 13.47 |
| | 95.0 | 95.6 | 38.97 | 95.6 | 38.97 | |

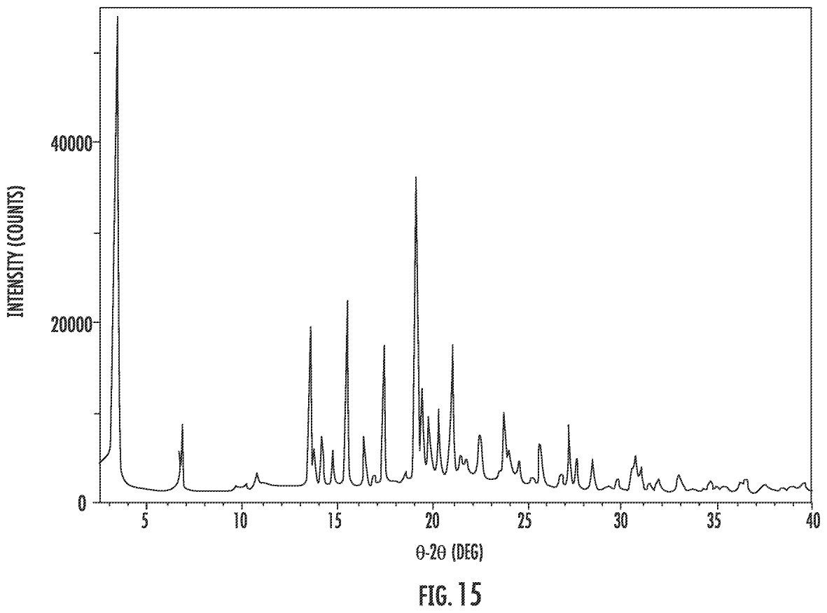

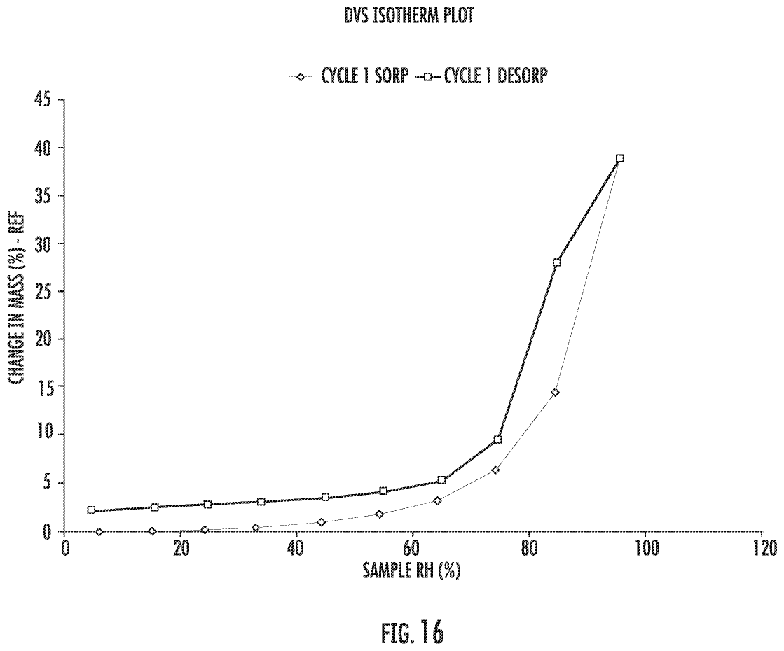

As described above in Table 10, and as illustrated in FIG. 16, following equilibration of the L-lysine gemfibrozil salt Material A at 5% relative humidity, the percent change in mass of the L-lysine gemfibrozil salt Material A began to increase at a relative humidity above 25%. From 5% to 25% relative humidity, the percent increase in mass of L-lysine gemfibrozil salt Material A during sorption was approximately 0.2% as determined using DVS. (FIG. 16 —Diamonds). From 5% to 85% relative humidity, the percent increase in mass of L-lysine gemfibrozil salt Material A during sorption was approximately 14.5%. The most significant water uptakes were observed above 85%; an additional, approximately 24.5% percent increase in mass of the L-lysine gemfibrozil salt Material A occurred from 85% to 95% relative humidity demonstrating the highly hygroscopic nature of the salt at these elevated relative humidities. This increase in percent mass may be even greater because the sample did not equilibrate within the time limits for these steps. FIG. 16 also demonstrates that following desorption from 95% to 5% relative humidity (FIG. 16—Squares), L-lysine gemfibrozil salt Material A retained approximately 2.1% of the gained moisture. Upon desorption, hysteresis is observed. In this example, the post-DVS sample deliquesced.

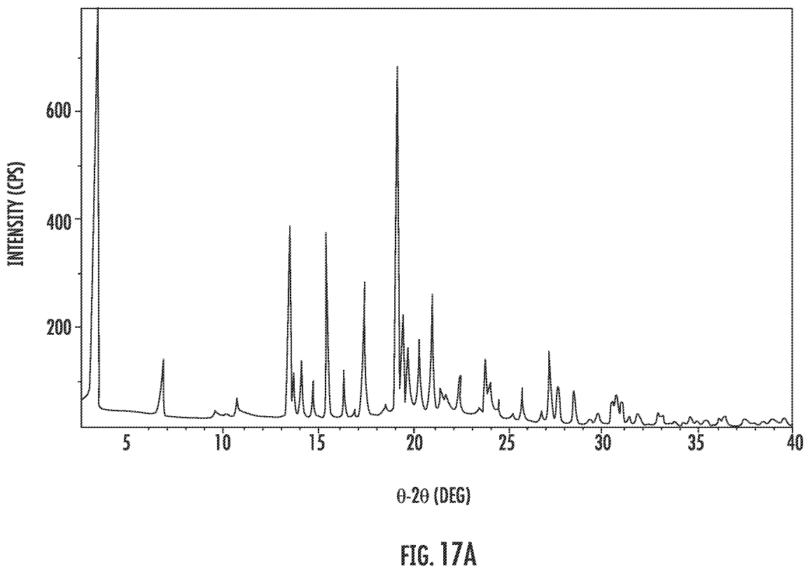

In an example, a post-DVS L-lysine gemfibrozil salt Material A, although sticky, did not experience a change in form as determined by its XRPD pattern. As illustrated in FIG. 17A, observed peaks by XRPD of a post-DVS L-lysine gemfibrozil salt Material A were consistent with an L-lysine gemfibrozil salt prepared prior to exposure to increased relative humidities. The observed peaks of the post-DVS salt were consistent with the pattern of peaks observed for an L-lysine gemfibrozil salt Material A prepared prior to exposure to increased relative humidities (FIG. 15). These data suggest that under these stressed conditions, L-lysine gemfibrozil salt Material A did not undergo a form change upon exposure to increased relative humidity.

In an example, the amount of L-Lysine gemfibrozil salt Material A was scaled-up during preparation. In an embodiment, the amount of L-Lysine gemfibrozil salt Material A was produced on at least a one gram scale. As illustrated in FIG. 17B, the observed peaks by XRPD of the scaled-up salt were consistent with the pattern of peaks for smaller scale production of the L-Lysine gemfibrozil salt Material A (FIG. 15). These data suggest that upon scaling up the amount of the salt produced, the same L-Lysine gemfibrozil salt Material A is formed as evidenced by the lack of form change determined by XRDP analysis.

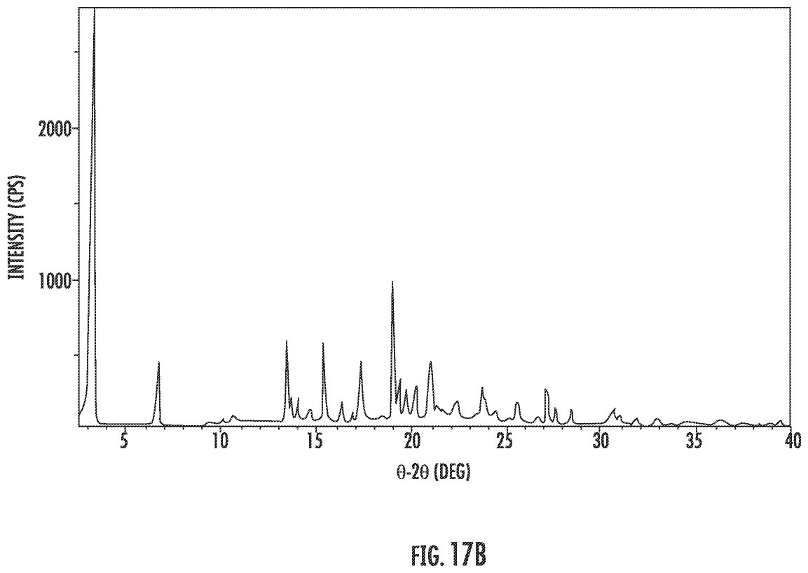

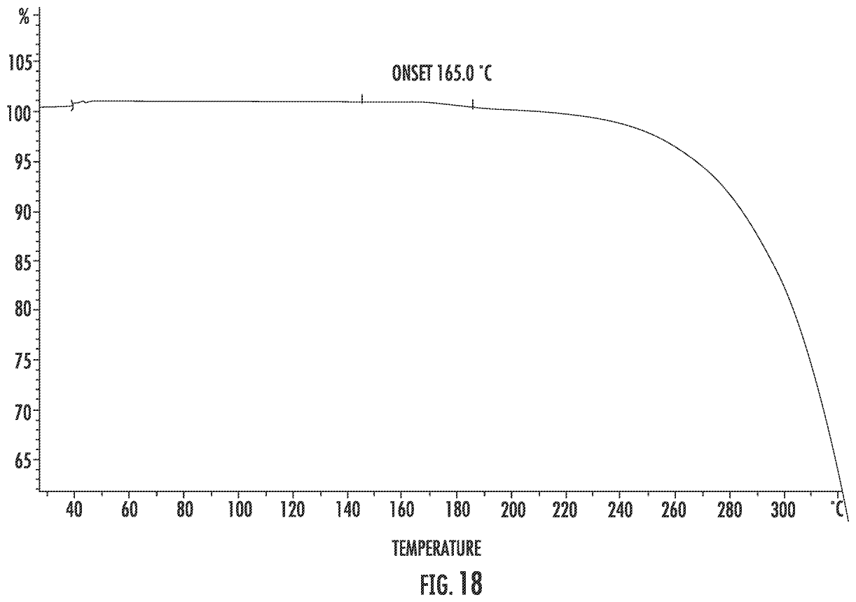

In an embodiment, an L-lysine gemfibrozil salt is substantially anhydrous and unsolvated, which may be determined using TGA. An exemplary L-lysine gemfibrozil salt, L-Lysine gemfibrozil salt Material A, does not experience weight loss at temperatures approximately below 165° C. as demonstrated by TGA. As illustrated in FIG. 18, the weight of L-Lysine gemfibrozil salt Material A does not decrease until approximately 165° C. as determined using TGA. This temperature may be the onset of decomposition.

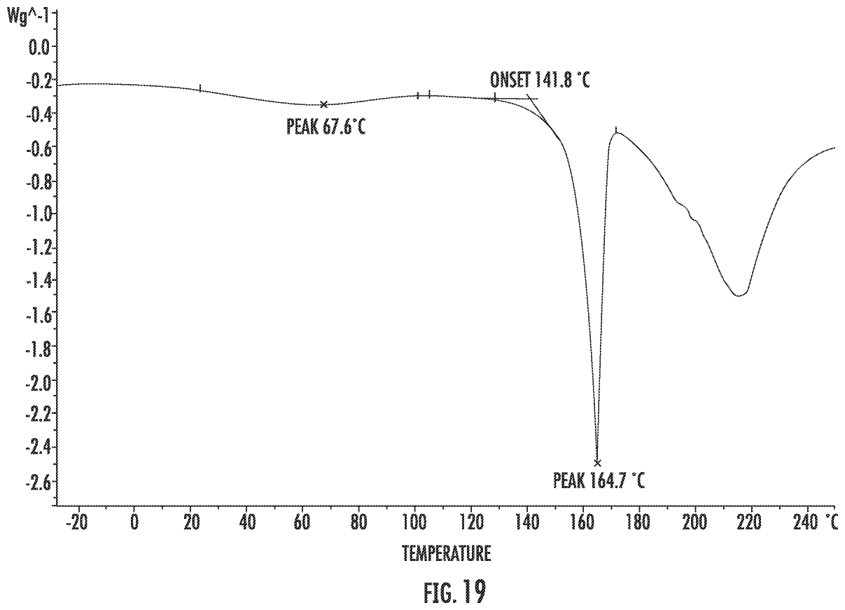

In an embodiment, L-lysine gemfibrozil salt Material A has a large endotherm peak as determined by DSC. In an embodiment, L-lysine gemfibrozil salt Material A has an onset of its large endotherm peak at approximately 142° C., which peaks at approximately 165° C. as illustrated in FIG. 19. This endotherm peak is concurrent with the onset of weight loss of L-lysine gemfibrozil salt Material A observed by TGA analysis in FIG. 18. The exemplary L-lysine gemfibrozil salt also has a small broad endotherm peak at 68° C., typically indicates the presence of volatiles, which contradicts with the TGA analysis showing no weight loss prior to approximately 165° C. The reason for this discrepancy in not known, but may be caused by inhomogeneity of the sample due to an uneven distribution of moisture throughout the solids.

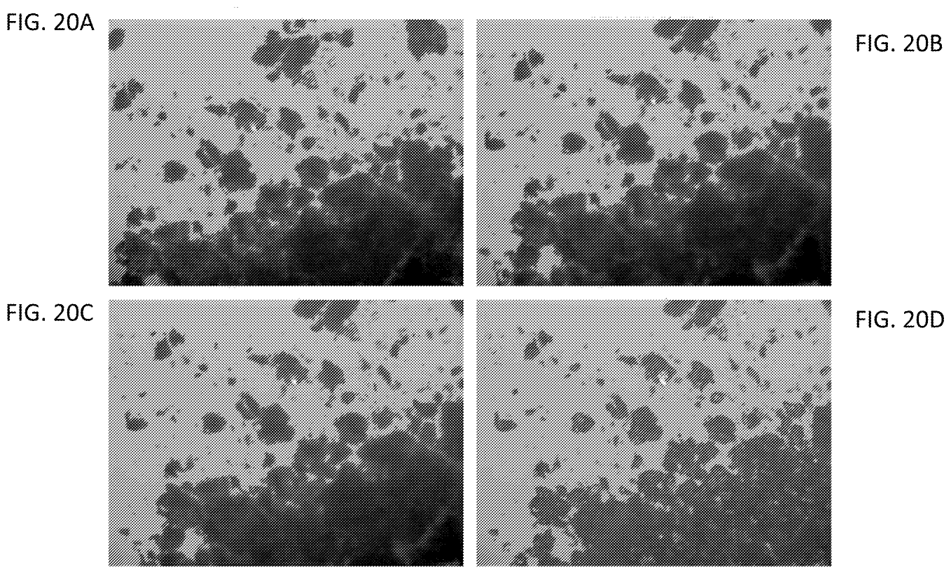

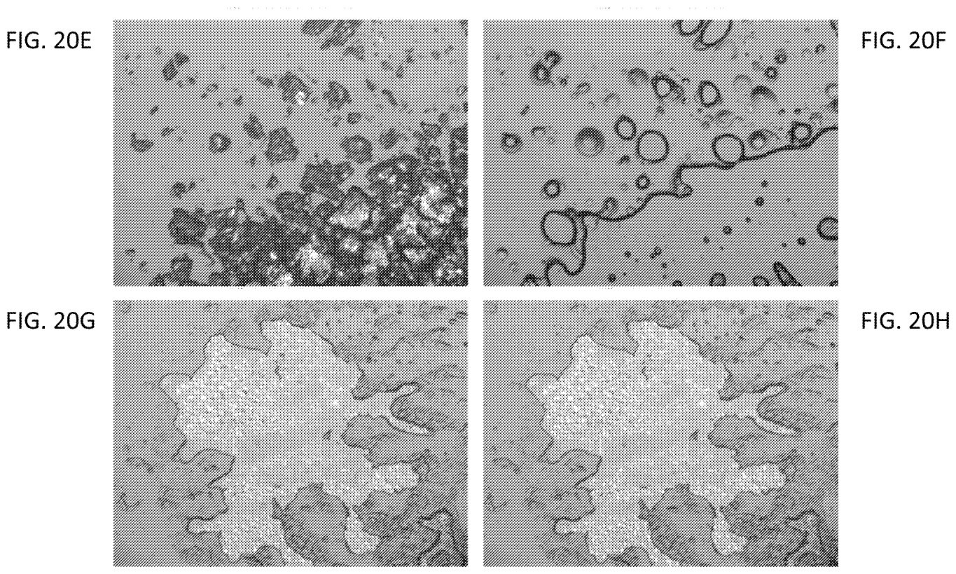

In an embodiment, L-lysine gemfibrozil salt Material A has a melting temperature starting at approximately 158.9° C. as determined by hot-stage microscopy. For example, L-lysine gemfibrozil salt Material A at ambient temperature is composed of crystallites and agglomerates as illustrated FIG. 20A. FIG. 20B illustrates the exemplary L-lysine gemfibrozil salt at 72.9° C., where some cracking in the crystallites is observed, possible due to loss of minor volatiles. FIG. 20C illustrates the exemplary L-lysine gemfibrozil salt at 145.8° C., where particles are beginning to soften. FIG. 20D illustrates the exemplary L-lysine gemfibrozil salt at 158.9° C., where the onset of melting is observed. FIG. 20E illustrates the exemplary L-lysine gemfibrozil salt at 162.4° C., where melting continues. FIG. 20F illustrates the exemplary L-lysine gemfibrozil salt at 168.0° C., where the salt is completely melted. This temperature is closely resembles the temperature observed with loss of weight during TGA analysis in the example illustrated in FIG. 18, as well as the temperature observed for the peak of the sharp endotherm observed during DSC in the example illustrated in FIG. 19. FIG. 20G illustrates the exemplary L-lysine gemfibrozil salt that was subsequently quenched to ambient temperature and recrystallized upon cooling. The sample was further reheated at a rate of 20° C./minutes until approximately 143° C. and then at a rate of 10° C./minute. As shown in FIG. 20H, at 88.3° C. during reheat, an increase in recrystallization was observed. No other events occurred until 163.3° C., the onset of melting. Melting was complete by 168.6° C. By 190.1° C., discoloration was observed, indicative of decomposition.

In an embodiment, a gemfibrozil L-lysine salt may have at least one of the following properties, including being freely soluble in an aqueous solution as determined by the equilibrium solubility values presented in Table 2 for the gemfibrozil L-lysine salt Material A; having a solubility of approximately 336 mg/ml in artificial saliva; having a solubility selected from 50 mg/ml increments selected from the range of 100 mg/ml to 1000 mg/ml; rapidly dissolve in an aqueous solution; rapidly dissolve in an aqueous solution as described in Table 4; having being more soluble in an aqueous solution than free-form gemfibrozil; having an equilibrium solubility selected from 50 mg/ml increments selected from the range of 400 mg/ml to 1000 mg/ml; a 1:1 mol:mol stoichiometry of gemfibrozil:L-lysine as determined by a ¹H NMR spectrum of the L-lysine gemfibrozil salt; having a single crystalline phase; having a crystalline phase having some degree of disorder; having a mixture of crystalline phases; having an XRPD pattern comprising observed peaks at angular position 2θ as shown in Table 9 (although the representative peaks or characteristic peaks of the L-lysine gemfibrozil salt may vary from the observed peaks of this example); having a consistent XRPD pattern between small-batch production versus scaled-up production of the gemfibrozil L-lysine salt; having a low hygroscopicity below approximately 25% relative humidity as determined using DVS; having a low hygroscopicity below approximately 25% relative humidity and experiencing increases in mass of approximately 0.2% between 5% to 25% relative humidity; having a low hygroscopicity below approximately 85% relative humidity; having a low hygroscopicity below approximately 85% relative humidity and experiencing increases in mass of approximately 14.5% between 5% to 85% relative humidity as determined using DVS; having a highly hygroscopic nature above between 85% to 95% relative humidity; having no change in form upon exposure to increased humidity as determined by DVS; having a consistent XRPD pattern between a pre-DVS gemfibrozil L-lysine salt and a post DVS gemfibrozil L-lysine salt following absorption and desorption as illustrated in FIG. 16; being substantially anhydrous and unsolvated; being substantially anhydrous and unsolvated as determined by TGA; not experiencing weight loss at temperatures below approximately 165° C. as determined by TGA; having an onset of decomposition at approximately 165° C.; having a peak endoderm during DSC at approximately 165° C.; having a large endoderm peak during DSC at approximately 142° C. which peaks at approximately 165° C.; and having broad endotherm peaks at 68° C. and 165° C. as determined by DSC; having an onset of melting occur at approximately 152° C. as determined by having a sharp endotherm during DSC; —having a melting temperature starting at approximately 159° C.; having a melting temperature starting at approximately 159° C. as determined by hot-stage microscopy; having a melting temperature starting at approximately 159° C. and continuing through 168° C. as determined by hot-stage microscopy; having a few volatiles present; and being an L-lysine gemfibrozil salt as described for L-lysine gemfibrozil salt Material A.

Instrumental Techniques

X-Ray Powder Diffraction (XRPD)—XRPD patterns were collected with a PANalytical X'Pert PRO MPD or PANalytical Empyrean diffractometers using an incident beam of Cu radiation produced using an Optix long, fine-focus source. An elliptically graded multilayer mirror was used to focus Cu Kα X-rays through the specimen and onto the detector. Prior to analysis, a silicon specimen (NIST SRM 640e) was analyzed to verify the observed position of the Si 111 peak is consistent with the NIST-certified position. A specimen of the sample was sandwiched between 3 μm thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 nm from the specimen and Data Collector software v. 5.5.

Computational Study (XRPD Indexing)—XRPD patterns were indexed using X'Pert High Score Plus software 2.2a (2.2.1). Successful indexing indicates that a sample is composed primarily of a single crystalline phase. Agreement between allowed peak positions and observed peaks indicates a consistent unit cell determination. No attempts at molecular packing were performed to confirm the tentative indexing solutions within the scope of this work.

Each of the gemfibrozil salts has an X-Ray Powder Diffraction (XRPD) pattern, which may be used to help identify the given form of the salt. Under most circumstances, peaks within the range of up to approximately 30 degrees 2θ were selected to assist in identification of the salt. Rounding algorithms were used to round each peak to the nearest 0.1 degree or 0.01 degree 20 depending on the instrument used to collect the data and/or the inherent peak resolution. The location of the observed peaks at angular position 2θ was determined using the proprietary software TRIADS™ and the wavelength used to calculate these spacings was the Cu-Ki wavelength. Peak position variabilities are given to within +/−0.2 degree 2θ. However, accuracy and precision associated with any particular measurement reported has not been determined. Measurements on independently prepared samples on different instruments may lead to variability which is greater than +/−0.2 degree 2θ.

Representative peaks of the sample may only be determined when multiple diffraction patterns are available in order to provide an assessment of particle statistics and/or particle orientation. If the particle statistics and/or particle orientation are determined to be negligible, then the XRPD pattern is representative of the powder average intensity for the sample, and prominent peaks may be identified as “Representative Peaks.” If they are not negligible, or if they are not available, the observed peaks may not necessarily be representative peaks for the gemfibrozil salt.

“Characteristic Peaks” are a subset of representative peaks and can be used to differentiate one crystalline polymorph from another crystalline polymorph. Characteristic peaks are determined by evaluating which representative peaks, if any, are present in one crystalline polymorph of a compound against all other known crystalline polymorphs of that compound to within +/−0.2 degree 2θ. In the examples described herein, the peaks illustrated in the figures are observed peaks and may or may not be representative peaks. Characteristics peaks may or may not be illustrated.

Proton Nuclear Magnetic Resonance Spectroscopy (¹H NMR)—The solution NMR spectra were acquired with an Agilent DD2-400 spectrometer. The samples were prepared in DMSO-d₆ containing TMS.

Thermogravimetric Analysis (TGA)—TG analysis was performed using a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. The data acquisition parameters for the thermogram are determined.

Differential Scanning Calorimetry (DSC)—DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin, and zinc. The adjustment is then verified with octane, phenyl salicylate, indium, tin, and zinc. The sample was placed into a hermetically sealed aluminum DSC pan, and the weight was recorded. The pan lid was pierced and then inserted into the DCS cell. A weighed aluminum pan configured as the sample pan was placed on the reference side of the cell. The pan lid was pierced prior to sample analysis.

Dynamic Vapor Sorption (DVS)—Moisture sorption/desorption data were collected on a SMS Intrinsic Dynamic Vapor Sorption Analyzer. Sodium chloride and PVP were used as calibration standard. Samples were not dried prior to analysis. Sorption and desorption data were collected over a range from 5% to 95% relative humidity at 10% relative humidity increments under a nitrogen purge. The equilibrium criterion used for analysis was less than 0.0100% weight change in 5 minutes with a maximum equilibration time of 3 hours. Data were not corrected for the initial moisture content of the samples.

Hotstage microscopy—Hot stage microscopy was performed using a Linkam hot stage (FTIR 600) mounted on a Leica DM LP microscope equipped with a SPOT Insight™ color digital camera. Temperature calibrations were performed using USP melting point standards. Samples were placed on a cover glass, and a second cover glass was placed on top of the sample. As the stage was heated, each sample was visually observed using a 20×/0.40 #566003 objective with a polarized light or a 10×/0.22 #556031 objective with polarized light. Images were captured using SPOT software (v. 4.5.9).

Polarized light microscopy—Light microscopy was performed using a Leica DM LP microscope equipped with a SPOT Insight™ color digital camera. Each sample was placed on a glass slide, a cover glass was placed over the sample, and a drop of mineral oil was typically added to cover the sample by capillarity. Each sample was observed using a 0.8-10× objective with polarized light.

Other methods known in the art may be used to determine and characterize the nature of the gemfibrozil salts referenced herein.

Filter Bias Evaluation Studies

Filter bias evaluation was performed to identify a suitable membrane for filtration of gemfibrozil. Polyvinylidene difluoride (PVDF), polytetrafluoroethylene (PTFE), GH Polypro (GHP), and 0.45 μm Nylon membrane filters were used. A standard solution of gemfibrozil (approximately 200 μg/mL) was analyzed for peak area HPLC before and after filtration. Of the membranes analyzed, all displayed recoveries with +/−0.5% of the unfiltered solution indicating no significant retention on filtration.

Dissolution Studies

Dissolution experiments using free-from gemfibrozil were performed using a VanKel VK7010 dissolution apparatus equipped with a VK750D heater/circulator. USP <711> dissolution apparatus 2 (paddle method) was used. Dissolution medium was 900 mL of artificial saliva, pH 7.30. The temperature was maintained at 37° C. The paddles were rotated at 100 RPM. Six-hundred milligrams of gemfibrozil was added into each vessel at t=0. Samples aliquots of 2 mL were taken at 0.25, 0.5, 1, 2, 3, and 4 hours and filtered immediately using a 0.45 μm Nylon membrane syringe filter, whereby the filtrate was diluted at a 1:1 ratio with methanol and then vortexed. Samples were analyzed by HPLC.

Dissolution experiments using the salt forms of gemfibrozil were performed using a VanKel VK7010 dissolution apparatus equipped with a VK750D heater/circulator. The 2 paddle method was used. Dissolution medium was 250 mL of artificial saliva, pH 7.30. The temperature was maintained at 37° C. The paddles were rotated at 50 RPM. Amounts of gemfibrozil salt equivalent to 100 milligrams of free-form gemfibrozil was added into each vessel at t=0. Samples aliquots of 2 mL were taken at 5, 10, and 15 minutes and filtered immediately using a 0.45 m Nylon membrane syringe filter. Samples were analyzed by HPLC.

Kinetic Solubilities/Visual Solubility Assessment—Weighed samples were treated with aliquots of solvent at ambient temperature unless otherwise indicated. Samples were typically sonicated between additions to facilitate dissolution. Complete dissolution was observed through visual inspection. Solubilities were calculated based on the total amount of solvent added to achieve complete dissolution and may be greater than the value reported due to incremental solvent addition and the inherent kinetics of dissolution. If dissolution was not observed, values were reported as “less than.” If complete dissolution was observed upon the first aliquot of solvent, values were reported as “greater than.” If complete dissolution was observed the samples may be described as “approximately” soluble to the concentration of gemfibrozil salt corresponding to the amount of solvent used that resulted in complete dissolution as determined visually. These values are approximate. These values may also over-represent the amount of solvent required given that solvent was added incrementally. Approximately 10 mg of gemfibrozil salt was weighed into a vial. Artificial saliva was added to the vial until complete dissolution was observed. Each volume addition was followed by vortex mixing or sonication and observation on the presence of solids was noted.

Equilibrium solubility of gemfibrozil salts in artificial saliva —Amounts of each gemfibrozil salt equivalent to 100 mg of free-form gemfibrozil were stirred in 1.5 mL of artificial saliva for 16 hours. Solutions were filtered using a 0.45 μm Nylon membrane. Quantification was performed using liquid chromatography.

Solubility descriptors were used as follows—Practically insoluble refers to a compound that is soluble at less than 0.1 mg/ml in solution. Very slightly soluble, refers to a compound that is soluble in an aqueous solution from 0.1 mg/ml to 1 mg/ml. Slightly soluble, refers to a compound that is soluble in an aqueous solution from 1 mg/ml to 10 mg/ml. Sparingly soluble, refers to a compound that is soluble in an aqueous solution from 10 mg/ml to 30 mg/ml. Soluble, refers to a compound that is soluble in an aqueous solution from 30 mg/ml to 100 mg/ml. Very soluble refers to a compound that is soluble in an aqueous solution at a concentration of greater than 1000 mg/ml. Freely soluble refers to a compound that is soluble in an aqueous solution from 100 mg/ml to 1000 mg/ml. Rapidly dissolving, refers to a compound that dissolves into solution within 5 minutes. In an embodiment, greater than 90% of an amount of gemfibrozil salt equivalent to 100 mg of gemfibrozil free form dissolves in artificial saliva within 5 minutes. In an embodiment, greater than 95% of an amount of gemfibrozil salt equivalent to 100 mg of gemfibrozil free form dissolves in artificial saliva within 5 minutes.

In an embodiment, artificial saliva comprises, consists essentially of, or consists of 0.0150% potassium chloride, 0.0117% sodium chloride, 0.2105% sodium bicarbonate, 0.2003% alpha-amylase, and 0.1020% mucin gastric, in a solution that is adjusted to have a pH of approximately 7.3. Other artificial saliva compositions having similar physiological properties may be used.

Other methods known in the art may be used to assess the solubility nature of the gemfibrozil salts referenced herein.

Salt Screen Methods

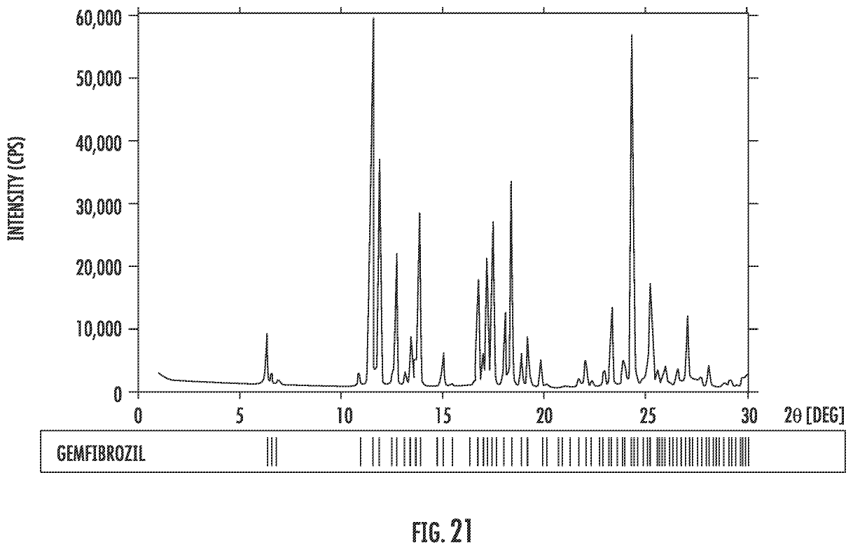

The gemfibrozil was purchased from Sigma Aldrich for use in this study. Lot SLBD3802V was characterized as received by XRPD and ¹H NMR to provide data for comparison with anticipated salts. In an example, XRPD data demonstrated that the XRPD pattern of lot SLBD3802V is crystalline and was successfully indexed indicating that the material was composed of a single crystalline phase as illustrated in FIG. 21. The crystalline phase has a monoclinic unit cell containing eight molecules of gemfibrozil. The volume of the unit cell, calculated from the indexing solution indicates that the material is likely unsolvated, but has additional space sufficient for accommodation of approximately one mole of water.

Other methods known in the art may be used to determine and characterize the nature of the gemfibrozil salts formed.

A salt screen was conducted with gemfibrozil and pharmaceutically acceptable salt formers. Salt formers previously reported to produce salts with gemfibrozil, including various alkyl amines, alkyl diamines, cycloalkyl amines, benzylamine, choline, tromethamine, and hydroxyl derivatives of t-butylamine, choline, adamantamine, and triethanolamine were not utilized in this screen. A variety of crystallization techniques and solvent systems were used in these salt formation experiments. Any isolated solids were examined by polarized light microscopy. Upon showing signs of crystallinity (i.e. birefringence and extinction), samples were analyzed by XRPD. XRPD patterns of generated materials were compared with each other, to the pattern of free-form gemfibrozil, and to the available patterns of salt formers. Solids exhibiting unique crystalline XRPD patterns were further characterized as described herein.

In an embodiment the pharmaceutically acceptable salt former was one of ethanolamine, L-arginine, L-lysine, sodium (from hydroxide and methoxide), potassium (from hydroxide), piperazine, meglumine, benzaathine, diethylamine, glucosamine, 4-(2-hydroxyethyl) morpholine (HEM), 1-(2-hydroxyethyl) pyrrolidine (HEP), magnesium hydroxide, or sodium bicarbonate.

In an embodiment the pharmaceutically acceptable salt former was one of ethanolamine, L-arginine, L-lysine, sodium (from hydroxide and methoxide), potassium (from hydroxide), or piperazine.

In an embodiment the pharmaceutically acceptable salt former was one of ethanolamine, L-arginine, or L-lysine.

### Examples of Methods

In an embodiment, ethanolamine is the pharmaceutically acceptable salt former. Formation of the ethanolamine gemfibrozil salt includes dissolving or suspending free-form gemfibrozil in a solvent followed by the addition of ethanolamine. In an embodiment, the ratio of ethanolamine to free-form gemfibrozil is 1:1.

In an embodiment, the solvent is hexanes. In an embodiment, following the addition of ethanolamine, diethyl ether is added along with stirring. A clarified gel may form. In an embodiment, stirring the composition for 2 days to 9 days at room temperature results in the generation of solids. In an embodiment, stirring the composition for 0.5 days, 1 day, 2 days, 3 days, 4 days. 5 days, 6 days, 7 days, 8 days, 9 days, 10 days, or 14 days results in the generation of solids. In an embodiment, stirring the composition for at least 0.5 days up until 14 days results in the generation of solids. In an embodiment, stirring the composition for 0.5 day increments selected from the range of 0.5 days to 14 days results in the generation of solids. These solids may be in the shape of aggregates of small needles and fines. These solids may be the ethanolamine gemfibrozil salt form. In an embodiment, the ethanolamine gemfibrozil salt is crystalline and demonstrates a unique XRPD pattern.

In an embodiment, L-arginine is the pharmaceutically acceptable salt former. Formation of the L-arginine gemfibrozil salt includes dissolving or suspending free-form gemfibrozil in a solvent followed by the addition of L-arginine. In an embodiment, the ratio of Iarginine to free-form gemfibrozil is 1:1. In an embodiment, the ratio of Iarginine to free-form gemfibrozil is 2:1.

In an embodiment, the solvent is a 50:50 mixture of acetonitrile:methanol. In such an embodiment, the solution is heated. The temperature of such heating is approximately 30° C., which may result in precipitation of solids. Additional solvent may be added and the solution is heated again until approximately 45° C. The sample is slow stirred at approximately 45° C. for approximately 2 days. The sample is slow cooled to room temperature resulting in the formation of solids. In such an embodiment, small particles of arginine Material A form. In such an embodiment, the L-arginine gemfibrozil salt (Material A), is crystalline and demonstrates a unique XRPD pattern. In an embodiment, the heating temperatures are varied. In an example, the heating temperature is 50° C. In an embodiment, the temperature is ambient temperature to 25° C.; 25° C. to 30° C., 30° C. to 35° C.; 35° C. to 40° C.; 40 to 45° C.; 45° C. to 50° C.; 50° C.-55° C.; or any interval between any of these temperature ranges. In an embodiment, the heating temperatures are selected from 1° C. increments selected from the range of 25° C. to 55° C. In an embodiment, the sample is slow stirred for approximately 0.5 days, 1 day, 2 days, 3 days, 4 days, 5 days, 6 days, 7 days, 10 days, or 14 days. In an embodiment, the amount of time being slow-stirred is at least 0.5 days up until 14 days. In an embodiment, the sample is slow stirred from 0.5 day increments selected from the range of 0.5 days to 14 days

In an embodiment, the solvent is a 25:75 mixture of isopropanol:water. In such an embodiment, a further 43:57 mixture of isopropanol:water may be added to the solution. In an embodiment, the ratio of isopropanol:water may vary by any integer between 25:75 to 43:57. In an embodiment, the ratio of isopropanol:water is selected from increments of 1 selected from the range of 25 to 43 for isopropanol and 75 to 57 for water so that the total adds up to 100. The solution is stirred for approximately 3 days at room temperature followed by fast evaporation. In an embodiment, the sample is stirred for approximately 0.5 days, 1 day, 2 days, 3 days, 4 days, 5 days, 6 days, 7 days, 10 days, or 14 days. In an embodiment, the amount of time being slow-stirred is at least 0.5 days up until 14 days. In an embodiment, the sample is slow stirred from 0.5 day increments selected from the range of 0.5 days to 14 days. The resulting sample forms of a gel. In such an embodiment, the sample is then be triturated with various antisolvents. In an embodiment, the antisolvent is at least one of diethyl ether or acetonitrile. The resulting solution is sonicated, and further stirred for approximately 1 day. In an embodiment, the sample is stirred for approximately 0.5 days, 1 day, 2 days, 3 days, 4 days, 5 days, 6 days, 7 days, 10 days, or 14 days. In an embodiment, the amount of time being slow-stirred is at least 0.5 days up until 14 days. In an embodiment, the sample is slow stirred from 0.5 day increments selected from the range of 0.5 days to 14 days. The sample is decanted and triturated with antisolvents as above. In such an embodiment, a slurry of arginine form A and arginine form B may form, whereby L-arginine is present. In an embodiment, the L-arginine gemfibrozil salt (Material A and Material B) is crystalline and demonstrates unique XRPD patterns.

In an embodiment, the solvent is acetonitrile. In such an embodiment, the solution is heated for approximately one hour at approximately 55° C. or any other amount of time up until approximately 2 days followed by which the sample is cooled to room temperature. In an embodiment, the sample is cooled from 1 hour increments selected from the range of 1 hour to 48 hours. In such an embodiment, a slurry of arginine Material A forms. In an embodiment, the free-form gemfibrozil in acetonitrile is heated for approximately one hour at 60° C. followed by which the sample is cooled to room temperature where the sample remains for up until approximately 7 days. In an embodiment, the sample remains for greater than 7 days, and up to any number of days in which the sample is stable. In an embodiment, a slurry of arginine form A forms, whereby L-arginine may be present. In an embodiment, the L-arginine gemfibrozil salt (Material A) is crystalline and demonstrates a unique XRPD pattern. In an embodiment, the heating temperatures are varied. In an example, the heating temperature is 50° C., 55° C., 60° C., or 65° C. In an embodiment, the temperature is ambient temperature to 25° C.; 25° C. to 30° C., 30° C. to 35° C.; 35° C. to 40° C.; 40 to 45° C.; 45° C. to 50° C.; 50° C.-55° C.; 55° C. to 60° C.; 60° C. to 65° C.; 65° C. to 70° C.; or any interval between any of these temperature ranges. In an embodiment, the heating temperatures is selected from 1° C. increments selected from the range of 25° C. to 70° C. In an embodiment, the sample is heated for approximately 20 minutes, 20 to 25 minutes; 25 to 30 minutes; 30 to 35 minutes; 35 to 40 minutes; 40 to 45 minutes; 45 to 50 minutes; 50 to 55 minutes; 55 minutes to 60 minutes; 1 hour to 2 hours; 2 hours to 3 hours; 3 hours to 4 hours; 4 hours to 5 hours; 5 hours to 10 hours; 10 hours to 24 hours; 1 day to 2 days; 2 days to 3 days; 3 days to 4 days; 4 days to 7 days; or any one minute interval between any of these times. In an embodiment, the amount of time being slow-stirred is at least 20 minutes up until 7 days. In an embodiment, the amount of time being slow-stirred is selected from 1 minute increments selected from the range of 1 minute to the minutes in 7 days.

In an embodiment, the solvent is methanol. In such an embodiment, the solution is heated at approximately 40° C. In an embodiment, the sample is heated to 25° C.; 25° C. to 30° C., 30° C. to 35° C.; 35° C. to 40° C.; 40 to 45° C.; 45° C. to 50° C.; 50° C.-55° C.; 55° C. to 60° C.; 60° C. to 65° C.; 65° C. to 70° C.; or any interval between any of these temperature ranges. In an embodiment, the heating temperature is selected from 1° C. increments selected from the range of 25° C. to 70° C. The sample is slow cooled to room temperature for approximately 4 days until the solution appears clear. In an embodiment, the sample is slow cooled to room temperature for any number of days until the solution appears clear. In an embodiment, the sample is slow stirred from 0.5 day increments selected from the range of 0.5 days to 7 days. Diethyl ether is then added followed by fast evaporation. In such an embodiment, the sample is triturated with isopropyl acetate and cyclopentyl methyl ether, or with hexanes. The sample is left at room temperature for approximately 8 days. In an embodiment, the sample remains for greater than 8 days, and up to any number of days in which the sample is stable. In such an embodiment, a slurry of arginine Material A forms, whereby L-arginine may be present. In an embodiment, the L-arginine gemfibrozil salt (Material A) demonstrates a unique XRPD pattern.

In an embodiment, L-lysine is the pharmaceutically acceptable salt former. Formation of the L-lysine gemfibrozil salt includes dissolving or suspending free-form gemfibrozil in a solvent followed by the addition of L-lysine. In an embodiment, the ratio of L-lysine to free-form gemfibrozil is 1:1.

In an embodiment, the solvent is a 20:80 mixture of ethanol:diisopropyl ether. In an embodiment, the ratio of ethanol:diisopropyl ether varies by any integer between 10:90 to 30:70. In an embodiment, the ratio of ethanol:diisopropyl ether is selected from increments of 1 selected from the range of 10 to 30 for ethanol and 90 to 70 for diisopropyl ether so that the total adds up to 100. In such an embodiment, a solid plug forms. Additional solvent is added to the solution. The sample is kept at room temperature for approximately 2 days forming a slurry. In such an embodiment, the slurry includes lysine form A In an embodiment, the L-lysine gemfibrozil salt (Material A) is crystalline with some degree of disorder and demonstrates a unique XRPD pattern. In an embodiment, the sample is kept at room temperature for approximately 1 day; 1 day to 2 days; 2 days to 3 days; 3 days to 4 days; 4 days to 7 days; or any one minute interval selected from the range of 0.5 days to 7 days, or up to any number of days in which the sample forms a slurry and is stable.

In an embodiment, the solvent is ethanol. In such an embodiment, the solution is stirred at room temperature for approximately 4 days followed by fast evaporation, which resulted in crystalline solids plus a gel. The sample is further triturated with diethyl ether and sonicated forming a solidified gel, then powdery solids. The slurry is left for approximately 5 days. In an embodiment, the sample is left for approximately 1 day; 1 day to 2 days; 2 days to 3 days; 3 days to 4 days; 4 days to 5 days; 5 days to 7 days; or any one minute interval selected from the range of 0.5 days to 7 days, or up to any number of days in which the sample is stable. In such an embodiment, the slurry includes lysine Material A. In an embodiment, the L-lysine gemfibrozil salt (Material A) is crystalline and demonstrates a unique XRPD pattern. The salt may have some degree of disorder.

In an embodiment, the solvent is acetonitrile. In such an embodiment, solids may persist. The sample is heated to 60° C. In an embodiment, the sample is heated to 25° C.; 25° C. to 30° C., 30° C. to 35° C.; 35° C. to 40° C.; 40 to 45° C.; 45° C. to 50° C.; 50° C.-55° C.; 55° C. to 60° C.; 60° C. to 65° C.; 65° C. to 70° C.; or any interval between any of these temperature ranges. In an embodiment, the heating temperatures are selected from 1° C. increments selected from the range of 25° C. to 70° C. In an embodiment, a few drops of water are added until solids clump together. The solids may be orange/yellow. Such solids are then broken apart forming a slurry. The heating of the slurry at approximately 60° C. is continued for approximately 3-4 hours and then cooled to room temperature. The solution is stirred for days forming a gel. In an embodiment, the solution is stirred for approximately 12 days. In an embodiment, the solution is stirred for 0.5 day increments selected from the range of0.5 days to 12 days. The solvent is decanted, and the remaining mixture is triturated with diethyl ether, and sonicated, repeating such a cycle at least two times resulting in the formation of powdery solids. The slurry is left at room temperature. In such an embodiment, the slurry may include lysine Material A. In an embodiment, the L-lysine gemfibrozil salt (Material A) is crystalline and demonstrates a unique XRPD pattern. The salt may have some degree of disorder.

Pharmaceutical Composition

An embodiment comprises, a pharmaceutical composition comprising, consisting essentially of or consisting of a gemfibrozil salt. In an embodiment, the pharmaceutical composition comprises, consists essentially of, or consists of at least one form of the gemfibrozil salt. In an embodiment, the pharmaceutical composition comprises, consists essentially of, or consists of two or more different gemfibrozil salt combinations. For example, any combination of an ethanolamine gemfibrozil salt with an L-arginine gemfibrozil salt and/or an Ir lysine gemfibrozil salt; any combination of an L-arginine gemfibrozil salt with an ethanolamine gemfibrozil salt and/or an L-lysine gemfibrozil salt; or any combination of an L-lysine gemfibrozil salt with an ethanolamine gemfibrozil salt and/or an L-arginine gemfibrozil salt. With the inclusion of other gemfibrozil salts, other combinations may be possible.

In an embodiment the gemfibrozil salt is formed from free-form gemfibrozil and a pharmaceutically acceptable salt former selected from at least one of ethanolamine, L-arginine, L-lysine, sodium (from hydroxide and methoxide), potassium (from hydroxide), piperazine, meglumine, benzathine, diethylamine, glucosamine HCl, 4-(2-hyroxyethyl) morpholine (HEM), 1-(2-hydroxyethyl) pyrrolidine (HEP), magnesium hydroxide and ethoxide, or sodium bicarbonate. In an embodiment the gemfibrozil salt is formed from free-form gemfibrozil and a pharmaceutically acceptable salt former selected from at least one of ethanolamine, L-arginine, or L-lysine.

In an embodiment the gemfibrozil salt is selected from at least one of ethanolamine gemfibrozil salt form A, I-arginine gemfibrozil salt Material A, L-arginine gemfibrozil salt Material B, L-lysine gemfibrozil salt Material A.

In an embodiment, the pharmaceutical composition comprises, consists essentially of, or consists of two or more forms of the gemfibrozil salt. For example, I-arginine gemfibrozil salt Material A and I-arginine gemfibrozil salt Material B, or L-lysine gemfibrozil salt Material A.

In an embodiment, the pharmaceutical composition further comprises at least one of a pharmaceutically acceptable carrier or a pharmaceutically acceptable excipient. In an embodiment, the pharmaceutically acceptable carrier or a pharmaceutically acceptable excipient may be suited for oral formulation. In an embodiment, the pharmaceutically composition may be in the form of an oral formulation.

In an embodiment, the pharmaceutically acceptable carrier may include any one or more agents selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, polyethoxylated castor oil, phosphate buffered saline (PBS), or any other suitable pharmaceutically acceptable carrier.

In an embodiment, the pharmaceutically acceptable excipient may include any one or more substances selected from the group consisting of an acidifying agent, an alkalizing agent, an antiadherent, an anticaking agent, an antifoaming agent, an antimicrobial preservative, an antioxidant, a binder, a coating agent, a color, a disintegrant, a diluent, an emulsifying agent, an extended release agent, a flavor, a glidant, a humectant, a lubricant, an ointment base, a preservative, a solubilizer, a sorbent, a sustained release agent, a sweetening agent, or any other suitable pharmaceutically acceptable excipient.

In an embodiment, the administering of the gemfibrozil salt or the pharmaceutical composition comprising the gemfibrozil salt may be any one or more of oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, and transmucosal. In an embodiment the gemfibrozil salt or the pharmaceutical composition comprising the gemfibrozil salt may be administered orally through at least one of a buccal, a sublingual, or a transmucosal route.

In an embodiment, the dose of gemfibrozil salt may be lower than a dose of free-form gemfibrozil that demonstrates similar efficacy in vivo in patients in need thereof. In an embodiment, total daily doses of oral free-form gemfibrozil for a titration period are indicated as follows:

| Time | Subject Weight | | |
|---|---|---|---|
| Point | 12-18 kg | 19-26 kg | ≥27 kg |
| Week 1 | 30 mg (2 mL) | 45 mg (3 mL) | 60 mg (4 mL) |
| Week 2 | 60 mg (4 mL) | 90 mg (6 ml) | 120 mg (8 mL) |
| Week 3 | 150 mg (10 mL) | 225 mg (15 mL) | 300 mg (20 mL) |
| Week 4 | 300 mg (20 mL) | 450 mg (30 mL) | 600 mg (40 mL) |
| Week 5 | 450 mg (30 mL) | 675 mg (45 mL) | 900 mg (60 mL) |

In an embodiment, a range of a gemfibrozil salt may be administered at 0.1-2000 mg/day. In an embodiment, a subrange of a gemfibrozil salt may be administered at 0.1-2000 mg/day having a low endpoint be any value selected from 0.1-1999 mg/day. The subrange may be any dose selected from 0.1 mg/day, 0.2 mg/day, 0.3 mg/day, 0.4 mg/day . . . , or 1999 mg/day in any 0.1 mg/day increment included in the subrange of 0.1-2000 mg/day. In an embodiment, a subrange of a gemfibrozil salt may be administered at 0.1-2000 mg/day having a high endpoint be any value selected from 0.2-2000 mg/day. The subrange may be any dose selected from 0.2 mg/day, 0.3 mg/day, 0.4 mg/day, 0.5 mg/day . . . , or 2000 mg/day in any 0.1 mg/day increment included in the subrange of 0.1-2000 mg/day.

In an embodiment, a range of a gemfibrozil salt may be administered at 0.1-1200 mg/day. In an embodiment, a subrange of a gemfibrozil salt may be administered at 0.1-1200 mg/day having a low endpoint be any value selected from 0.1-1199 mg/day. The subrange may be any dose selected from 0.1 mg/day, 0.2 mg/day, 0.3 mg/day, 0.4 mg/day . . . , or 1199 mg/day in any 0.1 mg/day increment included in the subrange of 0.1-1200 mg/day. In an embodiment, a subrange of a gemfibrozil salt may be administered at 0.1-2000 mg/day having a high endpoint be any value selected from 0.2-1199 mg/day. The subrange may be any dose selected from 0.2 mg/day, 0.3 mg/day, 0.4 mg/day, 0.5 mg/day . . . , or 1199 mg/day in any 0.1 mg/day increment included in the subrange of 0.1-1200 mg/day.

In an embodiment, the gemfibrozil salt may be administered once daily (QID). In an embodiment, the gemfibrozil salt may be administered twice daily (BID). In an embodiment, the gemfibrozil salt may be administered three times daily (TID). In an embodiment, the gemfibrozil salt may be administered on select days.

Method of Treating

Gemfibrozil salts, or pharmaceutical composition comprising gemfibrozil salts, may be used to treats any patient in need thereof. In an embodiment, a patient in need thereof may include a subject who needs to lower systemic triglyceride levels. In an embodiment, a patient in need thereof may also include a patient having late infantile neuronal ceroid lipofuscinosis type 2 (CLN2). In an embodiment, a patient in need thereof may also include a patient being treated with a form of a gemfibrozil tablet that has difficulty swallowing the tablet form of gemfibrozil.

Gemfibrozil salts, or pharmaceutical composition comprising gemfibrozil salts, may be used either alone or in combination with other CLN2 treatment drugs to enhance lysosomal-mediated waste clearance in certain cells in the brain.

### Embodiments of Detailed Description

1. A gemfibrozil composition comprising, consisting essentially of, or consisting of a gemfibrozil salt.

2. The gemfibrozil composition of embodiment 1, where the gemfibrozil salt has a solubility from 100 mg/ml to 1000 mg/ml in an aqueous solution.

3. The gemfibrozil composition of embodiment 1, where the gemfibrozil salt has a solubility of greater than 0.518 mg/mL, greater than 10 mg/mL, approximately 247 mg/mL, approximately 336 mg/mL, approximately 501 mg/mL, 247 mg/mL, 336 mg/mL, or 501 mg/mL in artificial saliva an aqueous solution.

4. The gemfibrozil composition of any one or more of the preceding embodiments, where the gemfibrozil salt dissolves in an aqueous solution within five minutes.

5. The gemfibrozil composition of any one or more of the preceding embodiments, where the aqueous solution is selected from water, a physiological buffer, or an artificial saliva.

6. The gemfibrozil composition of embodiment 5, where the aqueous solution is artificial saliva.

7. The gemfibrozil composition of embodiment 6, where the artificial saliva is a solution in water comprising 0.0150% potassium chloride, 0.0117% sodium chloride, 0.2105% sodium bicarbonate, 0.2003% alpha-amylase, and 0.1020% mucin gastric, with pH adjusted to 7.3.

8. The gemfibrozil composition of any one or more of the preceding embodiments, where the gemfibrozil salt is at least one of an ethanolamine gemfibrozil salt, an L-arginine gemfibrozil salt, or an L-lysine gemfibrozil salt.

9. The gemfibrozil composition of embodiment 8, where the gemfibrozil salt is an ethanolamine gemfibrozil salt.

10. The gemfibrozil composition of embodiment 9, where the ethanolamine gemfibrozil salt has an X-Ray Powder Diffraction (XRPD) pattern comprising peaks at angular position two theta of 8.94, 14.55, 14.70, 15.20, 15.49, 16.72, 17.01, 17.91, 21.34, and 22.14.

11. The gemfibrozil composition of at least one of embodiments 9 or 10, where the ethanolamine gemfibrozil salt comprises at least one property from the group consisting of low hygroscopicity, substantially anhydrous and unsolvated, gains up to 0.1% percent weight between a relative humidity of 5% to 55% as determined by dynamic vapor sorption (DVS), does not experience weight loss at temperatures below 140° C. as determined by thermogravimetric analysis (TGA), onset of decomposition begins at 140° C. as determined by differential scanning calorimetry (DSC), melting temperature of 62.9° C. to 65.2° C. as determined using hotstage microscopy, an equilibrium solubility in the aqueous solution of greater than 999 mg/mL, and a 1:1 mol:mol stoichiometry of gemfibrozil:ethanolamine as determined by ¹H NMR.

12. The gemfibrozil composition of any one or more of embodiments 9, 10, or 11, where the ethanolamine gemfibrozil salt is made by the steps of dissolving or suspending a free form of gemfibrozil in a mixture of hexanes, adding ethanolamine, adding diethyl ether, and stirring.

13. In an embodiment, the ethanolamine gemfibrozil salt is made by the steps of: dissolving or suspending a free form of gemfibrozil in a mixture of hexanes; adding ethanolamine; adding diethyl ether; and stirring.

14. The gemfibrozil composition of embodiment 8, where the gemfibrozil salt is an L-arginine gemfibrozil salt.

15. The gemfibrozil composition of embodiment 14, where the L-arginine gemfibrozil salt has an X-Ray Powder Diffraction (XRPD) pattern comprising peaks at angular position two theta of 5.36, 10.74, 12.02, 14.32, 17.69, 18.86, and 19.55.

16. The gemfibrozil composition of at least one of embodiments 14 or 15, where the L-arginine gemfibrozil salt comprises at least one property from the group consisting of low hygroscopicity, substantially anhydrous and unsolvated, gains up to 0.8% percent weight between a relative humidity of 5% to 85% as determined by DVS, does not experience weight loss at temperatures below 44° C. as determined by TGA, does not experience weight loss of greater than 0.1% at temperatures below 125° C. as determined by TGA, an onset of decomposition begins at 207° C. as determined by DSC, has a large sharp endotherm with a peak at 161° C. as determined by DSC, an equilibrium solubility in the aqueous solution of greater than 467 mg/mL, and has a 1:1 mol:mol stoichiometry of gemfibrozil:L-arginine as determined by ¹H NMR.

17. The gemfibrozil composition of any one or more of embodiments 14, 15, or 16, where the L-arginine gemfibrozil salt is made by the steps of: dissolving or suspending a free form of gemfibrozil in a 50:50 acetonitrile:methanol solvent; adding L-arginine; heating to 30° C.; adding additional 50:50 acetonitrile:methanol solvent; heating to 45° C.; stirring for 2 days; and slow cooling to ambient temperature.

18. The gemfibrozil composition of embodiment 8, where the gemfibrozil salt is an L-lysine gemfibrozil salt.

19. The gemfibrozil composition of embodiment 18, where the L-lysine gemfibrozil salt has an X-Ray Powder Diffraction (XRPD) pattern comprising peaks at angular position two theta of 3.36, 6.74, 13.50, 15.49, 17.42, 19.14, 19.43, 19.75, 20.31, and 21.04.

20. The gemfibrozil composition of at least one of embodiments 18 or 19, where the L-lysine gemfibrozil salt comprises at least one property from the group consisting of high hygroscopicity, substantially anhydrous and unsolvated, gains up to 2% percent weight between a relative humidity of 5% to 55% and gains up to 14.5% percent weight between a relative humidity of 5% to 85% as determined by DVS, does not experience weight loss at temperatures below 165° C. as determined by TGA, an onset of decomposition begins at 141.8° C. as determined by DSC, has a melting temperature of 158.9° C. to 168.0° C. as determined using hot-plate microscopy, an equilibrium solubility in the aqueous solution of greater than 534 mg/mL, and has a 1:1 mol:mol stoichiometry of gemfibrozil:L-lysine as determined by ¹H NMR.

21. The gemfibrozil composition of any one or more of embodiments 18, 19, or 20, where the L-lysine gemfibrozil salt is made by the steps of: dissolving or suspending a free form of gemfibrozil in a 20:80 ethanol:diisopropyl ether solvent; adding L-lysine; adding additional 20:80 ethanol:diisopropyl ether solvent; and stirring for 2 days.

22. The gemfibrozil composition of any one or more of embodiments 18, 19, or 20, where the L-lysine gemfibrozil salt is made by the steps of: dissolving or suspending a free form of gemfibrozil in an acetonitrile; adding L-lysine; heating to 60° C.; adding water; heating to 60° C. for 3 to 4 hours; cooling to ambient temperature; stirring; and decanting, triturating with diethyl ether, and sonicating multiple times.

23. The gemfibrozil composition of any one or more of embodiments 1 to 8, where the gemfibrozil salt is at least one of a sodium-gemfibrozil salt, a potassium gemfibrozil salt, or a piperazine-gemfibrozil salt.

24. A method for generating a gemfibrozil salt.

25. The method for generating the gemfibrozil salt of embodiment 24, comprising consisting essentially of, or consisting of: dissolving or suspending a free form of gemfibrozil in a solvent; and adding a pharmaceutically acceptable salt former.

26. The method for generating the gemfibrozil salt of embodiment 25, where the pharmaceutically acceptable salt former comprises at least one salt from the group consisting of ethanolamine, L-arginine, and L-lysine.

27. The method for generating the gemfibrozil salt of any one or more of embodiments 25 or 26, where the pharmaceutically acceptable salt former is ethanolamine and the gemfibrozil salt is an ethanolamine-gemfibrozil salt.

28. The method for generating the gemfibrozil salt of embodiment 27, where the solvent is a mixture of hexanes.

29. The method for generating the gemfibrozil salt of any one of embodiments 27 or 28, where following adding of the pharmaceutically acceptable salt former, the method further comprises: adding diethyl ether; and stirring, wherein solids comprising the ethanolamine-gemfibrozil salt form.

30. The method for generating the gemfibrozil salt of any one or more of embodiments 25 or 26, where the pharmaceutically acceptable salt former is L-arginine and the gemfibrozil salt is an L-arginine-gemfibrozil salt.

31. The method for generating the gemfibrozil salt of embodiment 30, where the solvent is a 50:50 acetonitrile:methanol solvent.

32. The method for generating the gemfibrozil salt of any one or more of embodiments 30 or 31, where following adding of the pharmaceutically acceptable salt former, the method further comprises: heating to 30° C.; adding additional 50:50 acetonitrile:methanol solvent; heating to 45° C.; stirring for 2 days; and slow cooling to ambient temperature, wherein solids comprising the L-arginine-gemfibrozil salt form.

33. The method for generating the gemfibrozil salt of any one or more of embodiments 30 or 31, where the pharmaceutically acceptable salt former is L-lysine and the gemfibrozil salt is an L-lysine-gemfibrozil salt.

34. The method for generating the gemfibrozil salt of embodiment 33, where the solvent is at least one of a 20:80 ethanol:diisopropyl ether solvent or acetonitrile.

35. The method for generating the gemfibrozil salt of any one or more of embodiments 33 or 34, where the solvent is a 20:80 ethanol:diisopropyl ether.

36. The method for generating the gemfibrozil salt of embodiment 35, where following adding of the pharmaceutically acceptable salt former, the method further comprises adding additional 20:80 ethanol:diisopropyl ether solvent and stirring for 2 days.

37. The method for generating the gemfibrozil salt of any one or more of embodiments 33 or 34, where the solvent is acetonitrile.

38. The method for generating the gemfibrozil salt of embodiment 37, where and following adding of the pharmaceutically acceptable salt former, the method further comprises: heating to 60° C.; adding water; heating to 60° C. for 3 to 4 hours; cooling to ambient temperature; stirring; and decanting, triturating with diethyl ether, and sonicating multiple times.

39. The method for generating the gemfibrozil salt of any one or more of embodiments 33 to 38, where a slurry comprising the L-lysine gemfibrozil salt forms.

40. The method for generating a gemfibrozil salt of any one or more of embodiments 24 to 39, where the aqueous solution is selected from water, a physiological buffer, or an artificial saliva.

41. A pharmaceutical composition comprising the gemfibrozil salt of any one or more of embodiment 1 to 23 or the gemfibrozil salt made by the method of any one or more of embodiment 24 to 40.

42. The pharmaceutical composition of embodiment 41, where further comprising at least one of a pharmaceutically acceptable carrier or a pharmaceutically acceptable excipient.

43. The pharmaceutical composition of embodiment 42, where the pharmaceutically acceptable carrier includes at least one agent selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, polyethoxylated castor oil, phosphate buffered saline (PBS), or any other suitable pharmaceutically acceptable carrier.

44. The pharmaceutical composition of any one or more of embodiments 42 or 43, where the pharmaceutically acceptable excipient includes at least one substance selected from the group consisting of an acidifying agent, an alkalizing agent, an antiadherent, an anticaking agent, an antifoaming agent, an antimicrobial preservative, an antioxidant, a binder, a coating agent, a color, a disintegrant, a diluent, an emulsifying agent, an extended release agent, a flavor, a glidant, a humectant, a lubricant, an ointment base, a preservative, a solubilizer, a sorbent, a sustained release agent, a sweetening agent, or any other suitable pharmaceutically acceptable excipient.

45. The pharmaceutical composition of any one or more of embodiments 41 to 44, where the pharmaceutical composition is in the form of an oral formulation.

46. A method of treating a subject having Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis and Neuronal Ceroid Lipofuscinoses including late infantile and juvenile form of disease comprising administering the gemfibrozil salt of any one or more of embodiment 1 to 23, the gemfibrozil salt made by the method of any one or more of embodiments 24 to 40, or the pharmaceutical composition of any one or more of embodiments 41 to 45 to the subject.

47. The method of treating according to embodiment 46, where the administering comprises a route of administration comprising one or more routes selected from the group consisting of oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, and transmucosal.

48. The method of treating, according to at least one of embodiments 46 or 47, where the gemfibrozil salt is administered in a range of 0.1-1200 mg/day.

While the present invention has been described in terms of its specific embodiments and specific examples, certain modifications and equivalents will be apparent to those skilled in the art and are intended to be included within the scope of the present invention.

1. A gemfibrozil composition comprising a gemfibrozil salt.

2. The gemfibrozil composition of claim 1, wherein the gemfibrozil salt has a solubility from 100 mg/ml to 1000 mg/ml in an aqueous solution, preferably wherein the gemfibrozil salt dissolves in aqueous solution within five minutes.

3. The gemfibrozil composition of claim 2, wherein the aqueous solution is selected from water, a physiological buffer, or an artificial saliva, preferably wherein the aqueous solution is the artificial saliva.

4. (canceled)

5. The gemfibrozil composition according to claim 3, wherein artificial saliva is a solution in water comprising 0.0150% potassium chloride, 0.0117% sodium chloride, 0.2105% sodium bicarbonate, 0.2003% alpha-amylase, and 0.1020% mucin gastric, with pH adjusted to 7.3.

6. The gemfibrozil composition according to claim 1, wherein the gemfibrozil salt is at least one of an ethanolamine gemfibrozil salt, an L-arginine gemfibrozil salt, or an L-lysine gemfibrozil salt.

7. The gemfibrozil composition according to claim 1, wherein the gemfibrozil salt is an ethanolamine gemfibrozil salt.

8. The gemfibrozil composition according to claim 7, wherein the ethanolamine gemfibrozil salt has an X-Ray Powder Diffraction (XRPD) pattern comprising peaks at angular position two theta of 8.94, 14.55, 14.70, 15.20, 15.49, 16.72, 17.01, 17.91, 21.34, and 22.14.

9. The gemfibrozil composition according to claim 1, wherein the gemfibrozil salt is an L-arginine gemfibrozil salt.

10. The gemfibrozil composition according to claim 9, wherein the L-arginine gemfibrozil salt has an XRPD pattern comprising peaks at angular position two theta of 5.36, 10.74, 12.02, 14.32, 17.69, 18.86, and 19.55.

11. The gemfibrozil composition according to claim 1, wherein the gemfibrozil salt is an L-lysine gemfibrozil salt.

12. The gemfibrozil composition according to claim 11, wherein the L-lysine gemfibrozil salt an XRPD pattern comprising peaks at angular position two theta of 3.36, 6.74, 13.50, 15.49, 17.42, 19.14, 19.43, 19.75, 20.31, and 21.04.

13. The gemfibrozil composition of claim 1, wherein:
the gemfibrozil salt is an ethanolamine gemfibrozil salt, and the ethanolamine gemfibrozil salt is made by the steps of:
dissolving or suspending a free form of gemfibrozil in a mixture of hexanes,
adding ethanolamine,
adding diethyl ether, and
stirring; or
the gemfibrozil salt is an L-arginine gemfibrozil salt, and the L-arginine gemfibrozil salt is made by the steps of:
dissolving or suspending a free form of gemfibrozil in a 50:50 acetonitrile:methanol solvent,
adding L-arginine,
heating to 30° C.,
adding additional 50:50 acetonitrile:methanol solvent,
heating to 45° C.,
and stirring for 2 days, and
slow cooling to ambient temperature; or
the gemfibrozil salt is an L-lysine gemfibrozil salt, and the L-lysine gemfibrozil salt is made by the steps of:
dissolving or suspending a free form of gemfibrozil in a 20:80 ethanol:diisopropyl ether solvent,
adding L-lysine,
adding additional 20:80 ethanol:diisopropyl ether solvent, and
stirring for 2 days, or
the L-lysine gemfibrozil salt is made by the steps of:
dissolving or suspending a free form of gemfibrozil in an acetonitrile,
adding L-lysine,
heating to 60° C.,
adding water,
heating to 60° C. for 3 to 4 hours,
cooling to ambient temperature,
stirring, and
decanting, triturating with diethyl ether, and sonicating multiple times.

14. A method for generating a gemfibrozil salt comprising:
dissolving or suspending a free form of gemfibrozil in a solvent; and
adding a pharmaceutically acceptable salt former,
wherein the pharmaceutically acceptable salt former comprises at least one salt from the group consisting of ethanolamine, L-arginine, and L-lysine.

15. The method for generating a gemfibrozil salt according to claim 14, wherein:
the pharmaceutically acceptable salt former is ethanolamine;
the gemfibrozil salt is an ethanolamine-gemfibrozil salt;
the solvent is a mixture of hexanes; and
following adding of the pharmaceutically acceptable salt former, the method further comprises:
adding diethyl ether and stirring,
wherein solids comprising the ethanolamine-gemfibrozil salt form.

16. The method for generating a gemfibrozil salt according to claim 14, wherein:
the pharmaceutically acceptable salt former is L-arginine;
the gemfibrozil salt is an L-arginine-gemfibrozil salt;
the solvent is a 50:50 acetonitrile:methanol solvent; and
following adding of the pharmaceutically acceptable salt former, the method further comprises:
heating to 30° C.;
adding additional 50:50 acetonitrile:methanol solvent;
heating to 45° C.;
stirring for 2 days; and
slow cooling to ambient temperature,
wherein solids comprising the L-arginine-gemfibrozil salt form.

17. The method for generating a gemfibrozil salt according to claim 14, wherein:
the pharmaceutically acceptable salt former is L-lysine;
the gemfibrozil salt is an L-lysine-gemfibrozil salt; and
the solvent is a 20:80 ethanol:diisopropyl ether solvent or acetonitrile, wherein
when the solvent is the 20:80 ethanol:diisopropyl ether solvent, following adding of the pharmaceutically acceptable salt former, the method further comprises adding additional 20:80 ethanol:diisopropyl ether solvent and stirring for 2 days; or
when the solvent is acetonitrile, following adding of the pharmaceutically acceptable salt former, the method further comprises:
heating to 60° C.;
adding water until solids clump together;
heating to 60° C. for 3 to 4 hours;
cooling to ambient temperature;
stirring; and
decanting, triturating with diethyl ether, and sonicating multiple times, wherein a slurry comprising the L-lysine gemfibrozil salt forms.

18. A pharmaceutical composition comprising the gemfibrozil salt of claim 1.

19. The pharmaceutical composition of claim 18, wherein the pharmaceutical composition is in the form of an oral formulation and further comprises at least one of a pharmaceutically acceptable carrier or a pharmaceutically acceptable excipient.

20. A method of treating a subject having Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis, and Neuronal Ceroid Lipofuscinoses comprising administering the gemfibrozil salt of claim 1.

21. The method of treating according to claim 20, wherein:
the administering comprises a route of administration comprising one or more routes selected from the group consisting of oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, and transmucosal; and.
the gemfibrozil salt is administered in a range of 0.1-1200 mg/day.
